(19) United States
(12) Patent Application Publication
(10) Pub. No.: US 2009/0227025 A1
(43) Pub. Date: Sep. 10, 2009

(54) Ex vivo human lung/immune system model using tissue engineering for studying microbial pathogens with lung tropism

(75) Inventors: Joan E. Nichols, Galveston, TX (US); Joaquin Cortiella, Galveston, TX (US); Ronald P. Mlcak, Bayou Vista, TX (US); Jean A. Niles, Galveston, TX (US)

Correspondence Address: ROBERT W STROZIER, P.L.L.C, PO BOX 429, BELLAIRE, TX, 77402-0429, US

(73) Assignee: THE BOARD OF REGENTS OF THE UNIVERSITY OF TEXAS SYSTEM

(21) Appl. No.: 11/352,924

(22) Filed: Feb. 13, 2006

Related U.S. Application Data
(63) Continuation-in-part of application No. PCT/US04/17940, filed on Jun. 7, 2004.
Application No. PCT/US04/17940 is a continuation-in-part of application No. 11/298,543, filed on Dec. 9, 2005.
(60) Provisional application No. 60/652,255, filed on Feb. 11, 2005.
(60) Provisional application No. 60/476,591, filed on Jun. 6, 2003.
(60) Provisional application No. 60/634,563, filed on Dec. 9, 2004.

(51) Int. Cl. C12N 5/02; C12Q 1/02; C12N 5/08

(52) U.S. Cl. 435/395; 435/29; 435/366

(57) ABSTRACT

A method for studying scaffold-based tissue engineering approaches in combination with the use of progenitor or stem cells to generate new lung tissue in an in vitro system. The engineered tissue system of this invention is used to monitor lung and immune system exposure of pathogen and/or toxins. The method involves growing engineered lung/immune tissue from progenitor cells in a bioreactor and then exposing the engineered lung/immune tissue to a pathogen and/or toxin. Once exposed, response of the engineered tissue is monitored to determine the effects of exposure to the immune component of the tissue and to lung component of the tissue. This invention also involves development of mixed engineered tissues including a first fully functional engineered tissue such as lung tissue and a second fully functional engineered tissued such as immune tissue from a single animal donor. The mixed systems can include more than two engineered tissues.

### RELATED APPLICATIONS

This application: (1) claims provisional priority to U.S. Provisional Patent Application Ser. No. 60/652,255, filed 11 Feb. 2005, (2) is a continuation-in-part of PCT Application PCT/2004/17940, filed 7 Jun. 2004 designating the United States and Nationalized U.S. patent application Ser. No. 10/559,219, filed 6 Dec. 2005, claiming provisional priority to U.S. Provisional Patent Application Ser. No. 60/476,591, filed 6 Dec. 2003 and (3) is a continuation-in-part of U.S. patent application Ser. No. 11/298,543, filed 9 Dec. 2005 claiming provisional priority to U.S. Provisional Patent Application Ser. No. 60/634,563, filed 9 Dec. 2004.

### BACKGROUND OF THE INVENTION

1. Field of the Invention

The present invention relates to ex vivo or engineered tissue systems including one or a plurality of tissue types and models using the systems to study responses of individual tissues and mixed tissues to pathogens and/or toxins and to study the progress of infections, dysfunctions, diseases and/or toxic responses of such individual and mixed tissues and methods for making and using same

More particularly, the present invention relates to ex vivo or engineered tissue systems including one or a plurality of tissue types such as lung and lymphatic tissues and models using the systems to study responses of individual tissues and mixed tissues to pathogens and/or toxins and to study the progress of infections, dysfunctions, diseases and/or toxic responses of such individual and mixed tissues, where the system includes stable engineered tissues having normal tissue histology, a pathogen/toxin delivery system and a monitoring system for monitoring, detecting and analyzing the tissue's response to a pathogen and/or a toxin and methods for making and using same.

2. Description of the Related Art

Currently, ex vivo systems for studying stem cell behavior have focused on animal models, with little concentration on developing ex vivo human stem cell systems.

Influenza virus infection causes three syndromes: (1) an uncomplicated rhinotracheitis (2) a respiratory viral infection followed by bacterial pneumonia, and (3) a viral pneumonia. Most studies that have attempted to correlate pathogenicity of various strains of influenza A virus with specific gene products have been done in animal models such as ferrets (1135, 136) or mice (4). Use of animal models in order to study human disease is difficult since the virulence of a given strain of virus reflects a complex series of interactions in which both host and virus-determined properties are involved. Host-determined factors such as immunological experience, major histocompatibility complex haplotype or activation of cells and induction of apoptosis may play a major role in determining the outcome of influenza virus infection in the host organism.

Recent studies looking at the tissue tropism of H5N1 infection in humans has shown that the disease in humans is not well defined but seems to be localized to both the intestine and the lower lung causing a primary viral pneumonia. The major site of replication for H5N1 in the human is the pneumocyte (same). But studies of cats infected with H5N1 have shown virus infection of both type I and type II pneumocytes, bronchiolar and bronchiole epithelial cells as well as smooth muscle cells of pulmonary branches. This might also be true of other potentially pandemic strains, which cause a severe viral pneumonia as part of the sequale of infection. Little is known about the specific sites in the lung that are permissive to influenza virus replication especially for H5N1 and other potentially pandemic avian or non-avian strains of influenza.

The clinical course of influenza viral pneumonia progresses rapidly. It can lead to hypoxemia and death within a few days of onset. Understanding the pathophysiology of this disease is aided by a thorough examination of the microscopic anatomy and the host response to infection. The alveolar epithelial cell and the capillary endothelial cell, maintain a tenuous interface between gas in the alveolar airspace and fluid in the capillary lumen. If an infection with influenza virus destroys alveolar epithelial cells, plasma leaks from the capillary, filling the airspace. If enough alveoli are involved, patient's respiration is severely impaired. This is especially true of patients with increased pulmonary capillary pressure (e.g., those with mitral stenosis), because destruction of alveolar epithelial cells will lead to greater extravasation of plasma and more pulmonary edema than in otherwise healthy people.

Recent reports have documented the identification of novel stem or progenitor cells exhibiting extraordinary plasticity from a variety of adult mammalian tissues (including fat, deciduous teeth, skin, muscle, and bone marrow that have given rise to multiple cell lineages. Relatively little is known about stem and progenitor cells that exist in the lung or the process of their differentiation and organization into lung tissue. However, several recent works have described potential sources of progenitor cells capable of generating some of the cellular components of lung tissue.

In one study, mesenchymal stem cells injected intravenously into lethally irradiated mice were shown to engraft into alveoli and bronchi and express lung-specific markers. Another study documented the ability of lung and bone marrow-derived cell populations with the Side Population (SP) phenotype to develop into lung alveolar epithelial cells. Historically, several epithelial cell types (including Clara cells, pulmonary neuroendocrine cells, basal cells, and type II pneumocytes, have been suggested to possess the potential to give rise to an array of lung-specific single cell lineages. It has also been suggested that pulmonary neuroendocrine cells or neuroendocrine bodies contribute to airway repair after injury and may also serve as a reservoir of progenitor cells capable of epithelial regeneration. But multipotent pulmonary stem or progenitor cells capable of differentiating into progeny with multiple differentiation phenotypes, including those cells with unipotent transient amplification potential, have not yet been identified for the lung. There has been some attempt by other researchers to develop three dimensional models of the lung using transformed cell lines or from fetal pulmonary cells, but the progress has been limited due to the complexity of the lung itself.

Thus, there is a need in the art for ex vivo or engineered tissues that can act as a platform for studying responses of individual engineered tissues and mixed tissue systems to pathogens, toxins or other environmental stresses difficult or impossible to test in vivo and to the construction of implantable ex vivo grown fully differentiated tissue.

### DEFINITIONS

Anti-N antibodies means antibodies that invoke an immune response to the nine (9) neuraminidase (N-1-N9) subtypes of the influenza A virus.

Anti-N1 antibodies means antibodies that invoke an immune response to the N1 subtype of the influenza A virus.

Anti-N2 antibodies means antibodies that invoke an immune response to the N2 subtype of the influenza A virus.

Anti-N3 antibodies means antibodies that invoke an immune response to the N3 subtype of the influenza A virus.

Anti-N4 antibodies means antibodies that invoke an immune response to the N4 subtype of the influenza A virus.

Anti-N5 antibodies means antibodies that invoke an immune response to the N5 subtype of the influenza A virus.

Anti-N6 antibodies means antibodies that invoke an immune response to the N6 subtype of the influenza A virus.

Anti-N7 antibodies means antibodies that invoke an immune response to the N7 subtype of the influenza A virus.

Anti-N8 antibodies means antibodies that invoke an immune response to the N8 subtype of the influenza A virus.

Anti-N9 antibodies means antibodies that invoke an immune response to the N9 subtype of the influenza A virus.

Anti-N1 antibodies means antibodies that invoke an immune response to the N1 subtype of the influenza A virus.

Anti-H antibodies means antibodies that invoke an immune response to the fifteen (15) haemagglutinin (H1-H15) subtypes of the influenza A virus.

Anti-H1 antibodies means antibodies that invoke an immune response to the H1 subtype of the influenza A virus.

Anti-H1 antibodies means antibodies that invoke an immune response to the H1 subtype of the influenza A virus.

Anti-H2 antibodies means antibodies that invoke an immune response to the H2 subtype of the influenza A virus.

Anti-H3 antibodies means antibodies that invoke an immune response to the H3 subtype of the influenza A virus.

Anti-H4 antibodies means antibodies that invoke an immune response to the H4 subtype of the influenza A virus.

Anti-H5 antibodies means antibodies that invoke an immune response to the H5 subtype of the influenza A virus.

Anti-H6 antibodies means antibodies that invoke an immune response to the H6 subtype of the influenza A virus.

Anti-H7 antibodies means antibodies that invoke an immune response to the H7 subtype of the influenza A virus.

Anti-H8 antibodies means antibodies that invoke an immune response to the H8 subtype of the influenza A virus.

Anti-H9 antibodies means antibodies that invoke an immune response to the H9 subtype of the influenza A virus.

Anti-H10 antibodies means antibodies that invoke an immune response to the H10 subtype of the influenza A virus.

Anti-H 11 antibodies means antibodies that invoke an immune response to the H11 subtype of the influenza A virus.

Anti-H12 antibodies means antibodies that invoke an immune response to the H12 subtype of the influenza A virus.

Anti-H13 antibodies means antibodies that invoke an immune response to the H13 subtype of the influenza A virus.

Anti-H14 antibodies means antibodies that invoke an immune response to the H14 subtype of the influenza A virus.

Anti-H15 antibodies means antibodies that invoke an immune response to the H15 subtype of the influenza A virus.

Anti-M antibodies means antibodies that invoke an immune response to the type-specific internal influenza virus matrix (M) protein.

FITC means the fluorescent tag Flourescein isothiocyanate.

Organoid means a discrete fragment of engineered tissue formed in in vitro culture.

### SUMMARY OF THE INVENTION

### General Engineered Tissue

The present invention relates to an engineered tissue system including a plurality of individual engineered tissues and one or more mixed engineered tissue including two or more individual engineered tissues, where the tissues are generated on a scaffold in a bio-reactor under microgravity conditions sufficient to allow 3D orientation and structural differentiation.

The present invention relates to a method for studying tissue responses including the step of isolating stem cells from an animal, differentiating and growing the isolated stem cells on a scaffold in a bio-reactor under microgravity conditions sufficient to allow 3D orientation and structural differentiation to form a plurality of stable, fully functional individual engineered tissues, constructing a mixed engineered tissue from two or more of the individual engineered tissues, exposing the mixed engineered tissue and its constituent individual engineered tissues to a pathogen, a toxin and/or an environmental stress, and monitoring the response of each tissue to the pathogen, toxin and/or environmental stress. The method can also include the step of intermittently, periodically or continuously exposing the tissues to the pathogen, toxin and/or environmental stress and monitoring the tissues to determine longer term tissue responses.

The present invention relates to a method for studying tissue responses including the step of isolating stem cells from an animal, differentiating and growing the isolated stem cells on a scaffold in a bio-reactor under microgravity conditions sufficient to allow 3D orientation and structural differentiation to form a plurality of stable, fully functional individual engineered tissues, constructing a mixed engineered tissue from two or more of the individual engineered tissues, exposing the mixed engineered tissue and its constituent individual engineered tissues to a pathogen and monitoring the response of each tissue to the pathogen. The method can also include the step of intermittently, periodically or continuously exposing the tissues to the pathogen and monitoring the tissues to determine disease propagation and progression.

The present invention relates to a method for studying a treatment including the step of isolating stem cells from an animal, differentiating and growing the isolated stem cells on a scaffold in a bio-reactor under microgravity conditions sufficient tot allow 3D orientation and structural differentiation to form a plurality of stable, fully functional individual engineered tissues, constructing a mixed engineered tissue from two or more of the individual engineered tissues, exposing the mixed engineered tissue and its constituent individual engineered tissues to a pathogen, a toxin and/or an environmental stress to form infected tissues, monitoring the response of each infected tissue to the pathogen, toxin and/or environmental stress, exposing the infected tissues to a treatment and monitoring the response of the tissues to the treatment. The method can also include the step of intermittently, periodically or continuously exposing the tissues to the treatment and monitoring the treated tissues to determine longer term treated tissue responses.

### Engineered Tissue Pathogen and Toxin Models

The present invention also provides an ex vivo engineered tissue (individual or mixed) model for studying pathogens and/or toxins, where the model includes engineered cells, tissues and/or organoids and/or nodes.

The present invention also provides a method for studying pathogens and/or toxins including exposing an engineered tissue (individual or mixed) model to a pathogen and/or toxin under controlled conditions, where the controlled conditions include pathogen and/or toxin exposure concentration, exposure time, medium properties or other factors that impact pathogenicity and/or toxicity.

The present invention also provides a method for monitoring progression of toxic responses, infections, dysfunctions or diseases, where the method includes the step of inducing a toxic response, infection, dysfunction and/or a disease in a engineered tissue (individual or mixed) model and monitoring a response of the model under controlled conditions, where the controlled conditions include pathogen and/or toxin exposure concentration, exposure time, medium properties or other factors that impact pathogenicity and/or toxicity.

The present invention also provides a method for monitoring treatments against toxins, infections, dysfunctions or diseases, where the method includes the step of inducing a toxic response, infection, dysfunction and/or a disease in a engineered tissue (individual or mixed) model, administering a treatment under treating conditions and monitoring a response of the model to the treatment, where the treating conditions include treatment concentration, if the treatment is a pharmaceutical, treatment intensity, if the treatment is not a pharmaceutical, exposure time, medium properties or other factors that impact pathogenicity and/or toxicity. The term pharmaceutical means any composition of matter that can be introduced into the engineered tissue or its environment.

### Engineered Lung Tissue

The present invention provides in vitro grown, engineered lung tissue derived from lung resident stem or progenitor cells, which normally function in repair and homeostasis of the lung, where the tissue is are capable of being utilized as in vitro engineered mammalian including human lung models in order to examine pathogensis of pathogen such as the influenza A virus or the toxicity of toxins such as nicotine. For additional information of engineered lung tissue, the reader is referred to U.S. patent application Ser. No. 11/298,543, filed 9 Dec. 2005, incorporated therein by reference.

The present invention also provides methods for examining cell types and protein products produced in long-term stable cultures of engineered lung epithelium tissue, and evaluating optimal conditions for infection of murine mammals and human engineered lung tissue with the influenza A virus. The present invention also relates to determining response similarities normal mammalian lung tissue and long-term stable engineered mammalian lung tissue.

The present invention also provides methods for using long-term, stable cultures of engineered lung tissue to study lung tissue responses to pathogens and/or toxins, to study long-term responses to one-time, intermittent, periodic or continuous exposure of the long-term stable cultures of engineered lung tissue to the pathogens and/or toxins and to study short-term and long-term treatments to both short-term and long-term exposure to the pathogens and/or toxins.

The present invention also provides methods for: (1) using engineered human models of the bronchiole-alveolar junction to study the development and function of the lung parenchyma, (2) developing a combined human lung/immune system tissue engineered model, (3) developing an upper respiratory tract model using similar methodologies, (4) evaluating combinations of existing biocompatible and biodegradable matrices or produce novel matrix materials that can be used to facilitate in vitro lung tissue development, (5) using the human model to develop a better understanding of human disease caused by microbial pathogens with lung tropism (including but not limited to influenza A pandemic and non-pandemic strains including H5N1 or other avian strains with pandemic potential), and (6) using the human model to develop better therapeutics for microbial agents with 1 (7) the use of the human model to develop therapeutics for any lung injury (pathogen, chemical, trauma) that can be tested in the system.

The present invention also relates to the use of the engineered tissue of this invention to examine lung tissue response to exposure to a pathogen in a manner that separates the response of the lung parenchyma from the response of the immune cells that populate the lung interstitium and bronchiole-alveolar lymph tissue (BALT). The innate non-leukocyte immune functions of lung epithelia play a significant role in the host response to microbial exposure. Activation of the innate response and the role of cytokines and antimicrobial peptides produced by lung epithelia to microbial exposure are not well understood. Airway epithelial cells express Toll-like receptors 1-10 and could provide for the recognition of a wide variety of microbial products. TLRs also play a role in the body's response to endogenous ligands including heat shock proteins and hyaluronic acid both of which are produced as a result of inflammation. In terms of the specific response of the lung to TLR signaling due to virus exposure, there is limited information available. TLR-3 had been shown to respond to stimulation with double stranded RNA. The response of airway and/or lung epithelia to microbial exposure can potentially include production of inflammatory mediators such as cytokines, leukotrienes and endothelians, as well as chemotactic mediators such as chemokines, defensins and cathelicidins but in many instances it is difficult to determine the in vivo innate response by the lung itself separate from the response of the leukocytes that populate the lung interstitium and alveolar space. The present invention also relates to evaluating the role of the lung parenchyma in the modulation of the lung proteome and the dynamic collection of specialized lung proteins, which influence the host's innate response to a microbial pathogen such as influenza A virus.

The present invention provides an ex vivo engineering lung tissue including immature lung cells isolated adult human stem cells from peripheral blood and grown in a rotary cell culture system that maintains the cells in a 3D orientation, where the cells are capable of differentiation into mature, fully functional lung tissue.

The present invention also provides ex vivo produced mature, fully functional engineered lung tissue.

The present invention also provides ex vivo or artificial lung tissue organoid.

The present invention provides methods for isolating, culturing and differentiating the immature lung stem or progenitor cells into mature, fully functional lung tissue. The present invention allows for the evaluation of the innate response of cells comprising the lung parenchyma (lung cells) without the response of resident leukocytes or lymphocytes which is not possible when looking at live animals or human explant specimens.

The present invention also provides methods for using the immature lung cells, mature, fully functional lung tissue or organoids to study human lung tissue response to pathogens or to other environmental stresses difficult to study in vivo.

The present invention also provides methods for pre-sensitizing cells, mature, fully functional lung cells, tissue or organoids to pathogens or to other environmental stresses difficult to sensitize in vivo.

The present invention provides implantable ex vivo grown, mature, fully lung cells, tissues or organoids.

Progenitor Populations Developed into Complex Tissues in the Lung

The invention provides that a somatic lung progenitor cell population capable of support lung tissue development in both in vitro and in vivo models, where the cells can be differentiated into numerous cell types that produce Clara cell protein 10 (CC10), cytokeratin, and surfactant protein C(SP-C) prior to formation of cell/polymer constructs. The present invention also provides long-term (4-8 week) in vitro stable cultures of tissue engineered lung epithelium as human lung models for pathogenesis studies using influenza A virus and/or other pathogens and/or toxins.

### Innate Responses of the Cells of the Bronchiole-Alveolar Junction

The present invention also provide engineered tissue is capable of producing anti-microbial peptides called defensins and cathelicidins, which are innate immune factors present in airway surface liquid and make up part of the lung's natural defenses. These peptides are produced by several different cell types in the lung and respiratory tract including airway epithelial cells, macrophages and neutrophils. The cultures of pulmonary endothelial cells of this invention produce prostaglandins, when properly stimulated in the presence of neutrophils, leukotriene. The cultured airway epithelial cells of this invention are also capable of synthesizing both cyclooxygenase and lipoxygenase products. Engineered type II pneumocyte cells are capable of the de novo metabolism of arachidonic acid to both cyclooxygenase and lipoxygenase products and the production of leukotrienes is dependent on both time in culture and agonist. Thus, engineered tissue of alveolar type II cells of this invention are a potential source for these products.

### Engineered Lymphopietic Tissue

The present invention provides an ex vivo hemaotopoietic system including immature lymphopoietic cells isolated adult human stem cells from peripheral blood and grown in a rotary cell culture system that maintains the cells in a 3D orientation, where the cells are capable of differentiation into mature, fully functional T lymphocytes, B lymphocytes, NK lymphocytes and/or ex vivo lymphatic tissue or nodes. For additional details on the production of engineered lymphatic tissue, the reader is referred to co-pending U.S. patent application Ser. No. 10/559,219, filed 6 Dec. 2005, incorporated therein by reference.

The present invention also provides ex vivo produced mature, fully functional T lymphocytes, B lymphocytes, and/or NK lymphocytes.

The present invention also provides ex vivo or artificial lymphatic tissue or nodes.

The present invention provides methods for isolating, culturing and differentiating the immature lymphopoietic cells into mature, fully functional T lymphocytes, B lymphocytes, NK lymphocytes and/or ex vivo lymphatic tissue or nodes.

The present invention also provides methods for using the immature lymphopoietic cells, mature, fully functional T lymphocytes, B lymphocytes, NK lymphocytes and/or ex vivo lymphatic tissue or nodes to study human immune response to pathogens or to other environmental stresses difficult to study in vivo.

The present invention also provides methods for pre-sensitizing cells, mature, fully functional T lymphocytes, B lymphocytes, NK lymphocytes and/or ex vivo lymphatic tissue or nodes to pathogens or to other environmental stresses difficult to sensitize in vivo.

The present invention provides implantable ex vivo grown, mature, fully functional T lymphocytes, B lymphocytes, NK lymphocytes and/or ex vivo lymphatic tissue or nodes.

The present invention provides pre-sensitized, implantable ex vivo grown, mature, fully functional T lymphocytes, B lymphocytes, NK lymphocytes and/or ex vivo lymphatic tissue or nodes.

### DESCRIPTION OF THE DRAWINGS

The invention can be better understood with reference to the following detailed description together with the appended illustrative drawings in which like elements are numbered the same:

FIG. 1 depicts characterization of immature human progenitor cell populations evidencing negative data for CD45 and MHC class I and highly positive data for CD34, CD117 (c-kit), CD135 (fms-like tyrosine kinase-3 (flt-3);

FIG. 2 depicts differentiation trends of human progenitor cell populations as evidence by tracking β-tubulin production, NEUN-neuroenolase production, CC1-Clara Cell Protein 10 kd production, and SP A-Surfactant protein A production at days 1, 4, 7 and 14;

FIGS. 3A&B depict scanning electron micrograms of in vitro engineered lung progenitor cells growing on a polyglycolic acid (PGA) fiber of a PGA scaffold;

FIG. 4A depicts a photograph of a bioreactor cell containing engineered lung tissue after 4 week of growth;

FIG. 4B depicts pro-surfactant protein C (pro-SPC), the intracellular non secreted form of surfactant protein C, production in murine engineered lung tissue after 4 weeks of growth evidenced by the green color in this photograph;

FIG. 4C depicts is a confocal microscope photograph indicating the presence of type II pneumocytes in human TE lung organoids after 4 weeks of growth by the blue and green colors in the photograph;

FIGS. 5A-C depict one large confocal microscope picture of normal lung tissue after DAPI nuclear staining and two expanded views of a section of the normal lung tissue;

FIG. 6A depicts a confocal microscope picture of TE human lung tissue demonstrating the presence of type I pneumocytes: AQ-5 is red and pro-SPC is green;

FIG. 6B depicts a confocal microscope picture of TE human lung tissue demonstrating the presence of type I pneumocytes: AQ-5 is green;

FIGS. 7A&B depict confocal microscope pictures of TE murine lung tissue demonstrating the presence of the secreted from of surfactant protein A, relative to a control;

FIGS. 7C&D depict confocal microscope pictures of TE murine lung tissue demonstrating the presence of the Clara cell protein 10, relative to a control;

FIG. 8A depicts a confocal microscope picture of TE human lung tissue demonstrating the presence of the secreted from of surfactant protein A, relative to a control;

FIG. 8B depicts a confocal microscope picture of TE human lung tissue demonstrating the presence of the Clara cell protein 10, relative to a control;

FIGS. 9A-C depict confocal microscope pictures of human and murine TE engineered lung tissue after 6-8 weeks of growth demonstrating some expression of CD31, an endothelial cell marker;

FIGS. 10A&B depict a graphic of influenza A virus infection of a cells and scanning electron microgram of actual cells infected with fluorescent influenza A virus;

FIGS. 11A-D depict scanning electron micrograms of TE organoid 2 hours after viral exposure;

FIGS. 12A-F depict TE organoid responses to various antiviral agents;

FIGS. 13A-C depict SDS PAGE gel electrophoresis of immunoprecipitated proteins showing levels of tubulin, surfactant protein A and surfactant protein D at 4 to 8 weeks of growth;

FIGS. 14A-D depict TE-leukocyte cocultures, with sham primed leucocytes showed no specific response to influenza A by the lung associated immune cells;

FIGS. 14E-H depict leucocytes primed in vitro with heat killed influenza A/Marton/43 showing that a small portion of the T-lymphocytes are activated by exposure to live virus; and

FIG. 14I depict an expanded image of the FIG. 14H.

### DETAILED DESCRIPTION OF THE INVENTION

The inventors have demonstrated the development of engineered tissues (TE) including in vitro bone marrow, trachea and lung from mammalian (human, murine, ovine) adult stem cells. In an attempt to better understand the events in normal lung tissue regeneration, we have focused on isolation, characterization and differentiation of cells obtained from adult lung tissue. We have documents the existence in adult lung tissue of a population of pluripotent or multipotent progenitor cells, which are capable of generating complex engineered lung tissue when combined with a synthetic scaffold. Here, we emphasize the potential of scaffold-based tissue engineering approaches in combination with the use of progenitor or stem cells to generate new lung tissue in an in vitro system. In a variety of other tissue engineering applications, tissue assembly by cells has been facilitated by the use of polymer scaffolds which act as templates for cell-cell organization.

We isolated a somatic stem cell population from an adult mammalian tissue like the lung. Second, we differentiated the isolated somatic lung progenitor cells (SLPCs) into numerous cell types including, but not limited to, smooth muscle and mature cells producing neuron-specific enolase (NEUN), clara cell protein 10 (CC10) and surfactant protein C(SP-C) which is a secreted product of type II pneumocytes, when appropriate growth factor combinations were supplied to the differentiating cells. Third, we facilitated tissue assembly in vitro using a synthetic polymer scaffolds comprising polyglycolic acid (PGA) and Pluronic-F-127 (PF-127).

The fully functional lung tissue can also be constructed using the method used for the lymphatic tissue. The inventors have also produced data that has shown that exposure of MNL cultures to influenza virus induces apoptosis of lymphocytes. The major mechanism of apoptosis induction after influenza virus exposure is Fas-FasL. It is also possible that non-virus directed responses are suppressed in humans in part due to the induction of apoptosis after IAV-exposure and this may contribute to the development of secondary bacterial pulmonary infections. Lysates from Virus-Exposed NL. Autoradiograms of immunoprecipitated lysates using anti-N, anti-N2, anti-H1, anti-H3 or anti-M antibodies from monocyte-macrophages that were sham-exposed or exposed to influenza A strains A/Marton/43, wild type Bethesda/85, A/Mallard/NY6750/78 X Bethesda/85, A/Ann Arbor/6/60 X Bethesda/85, A/Kawasaki/87, A/Mallard/NY6750/78 X Kawasaki/87, A/Ann Arbor/6/60 X Kawasaki/87. ROLE OF NA IN THE INDUCTION OF CASPASE-3 IN CD3+ T LYMPHOCYTES. Results from five experiments. Apoptosis was significantly reduced in virus-exposed, monocyte-macrophage-depleted cultures at 24 (P=0.00302) and in NA-expressing cell-depleted cultures 24 (P=0.00022) hours after exposure. For each sample, data from 10,000 CD3+ cells were collected.

The inventors have previously found that an ex vivo hemaotopoietic system including immature lymphopoietic cells isolated adult human stem cells from peripheral blood and grown in a rotary cell culture system that maintains the cells in a 3D orientation, where the cells are capable of differentiation into mature, fully functional antigen naive T lymphocytes, B lymphocytes, NK lymphocytes and/or ex vivo lymphatic tissue or nodes. The inventors have also found the ex vivo engineered lymphatic tissue or nodes can be matured from immature lymphatic cells. The inventors have also found that methods for isolating, culturing and differentiating immature lymphopoietic cells can be practiced to produce mature, fully functional T lymphocytes, B lymphocytes, NK lymphocytes and/or ex vivo lymphatic tissue or nodes that the immature lymphopoietic cells, mature, fully functional T lymphocytes, B lymphocytes, NK lymphocytes and/or ex vivo lymphatic tissue or nodes can be used to study human immune response to pathogens or to other environmental stresses difficult to study in vivo. The inventors have also found that implantables can be constructed from the ex vivo grown, mature, fully functional T lymphocytes, B lymphocytes, NK lymphocytes and/or ex vivo lymphatic tissue or nodes.

The inventors have also found that an engineered tissue system of this invention can be used to monitor lung and immune system exposure of pathogen and/or toxins. The method involves growing engineered lung/immune tissue from progenitor cells in a bioreactor and then exposing the engineered lung/immune tissue to a pathogen and/or toxin. Once exposed, the response of the engineered tissue is monitored and analyzed to determine the effects of exposure to the immune component of the tissue and to the lung component of the tissue. In this way, the effects of infections, diseases and dysfunctions of the lung can be studies as can treatments of the infections, diseases and dysfunctions of the lung to ascertain their mode of action and their effectiveness.

This invention involves the development of mixed engineered tissues including a first fully functional engineered tissued such as lung tissue and a second fully functional engineered tissued such as immune tissue from a single animal donor. The mixed systems can obviously include more than two engineered tissues. The mixed systems can be studied independently from their constituents. However, by studying the mixed system and its constituents, individual and collective responses can be determined. Moreover, pharmaceutical screening can be greatly facilitated because using a combinational approach, candidate pharmaceutical compounds can be screened in each engineered tissued and in the mixed system to ensure that the candidates have an intended individual activity or collective activity.

The systems of this invention can be used to create new treatment modalities (vaccines, drug therapies, immune-based treatments, i.e., cellular or humoral based). This system can be used to study the development of cancers effecting tissues and organs in vitro. This system can be used to evaluate disease pathogenesis after microbial exposure as well as the influence on HLA subtypes, age, race, sex on the individual host response. The system can be used to study the development and progression of diseases. This system can be used to study human tissue and/or organ responses to radiation, chemotherapy, pharmaceutical therapy, chemical pathogen, toxins, biological exposure, and/or exposure to other environmental stresses.

Cell sources include, without limitation, lung progenitor cells derived from mammalian lungs, lung cells derived from other adult stem cells (bone marrow, peripheral blood, umbilical cord blood, wharton's jelly in the umbilical cord or from placental tissues) and lung cells derived from embryonic stem cells or any other source of stem cells that can be differentiated into lung tissue or the tissue of interest.

Although the inventors have demonstrated the production of individual engineered lung and lymphatic tissues and mixed lung/lymphatic tissues, the methodology used herein is directly applicable to any other individual tissue of interest and to any mixed system of interest. Thus, the methodology can be directed at prepare engineered tissues from any tissue in an animal or collection of tissues from the same animal or members of its species and to mixed system of these tissues. These individual and mixed engineered tissues can the serve as models to study individual and collective tissue responses to any pathogen, toxin, and/or stress.

Exemplary cell and tissues type that are capable of being engineered into stable, fully functional engineered tissue from suitable stem cells include, without limitation: (1) Keratinizing epithelial cells such as Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, and Hair matrix cell (stem cell); (2) Wet stratified barrier epithelial cells such as, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, and Urinary epithelium cell (lining urinary bladder and urinary ducts); (3) Exocrine secretory epithelial cells such as, Salivary gland mucous cell (polysaccharide-rich secretion), Salivary gland serous cell (glycoprotein enzyme-rich secretion), Von Ebner's gland cell in tongue (washes, taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), and Clara cell of lung; (4) Hormone secreting cells such as, Anterior pituitary cells (Somatotropes, Lactotropes, Thyrotropes, Gonadotropes, Corticotropes), Intermediate pituitary cell—secreting melanocyte-stimulating hormone, Magnocellular neurosecretory cells (secreting oxytocin, secreting vasopressin), Gut and respiratory tract cells secreting serotonin (secreting endorphin, secreting somatostatin, secreting gastrin, secreting secretin, secreting cholecystokinin, secreting insulin, secreting glucagon, secreting bombesin), Thyroid gland cells (thyroid epithelial cell, parafollicular cell), Parathyroid gland cells (Parathyroid chief cell, oxyphil cell), Adrenal gland cells (chromaffin cells, secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, and Mesangial cell of kidney; (5) (Gut, Exocrine Glands and Urogenital Tract) such as Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, and Epididymal basal cell; (6) Metabolism and storage cells such as Hepatocyte (liver cell), White fat cell, Brown fat cell, Liver lipocyte; (7) Barrier function cells (Lung, Gut, Exocrine Glands and Urogenital Tract) such as Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.); (8) Epithelial cells lining closed internal body cavities such as Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, and Corneal endothelial cell; (9) Ciliated cells with propulsive function such as Respiratory tract ciliated cell, Oviduct ciliated cell (in female), Uterine endometrial ciliated cell (in female), Rete testis cilated cell (in male), Ductulus efferens ciliated cell (in male), and Ciliated ependymal cell of central nervous system (lining brain cavities); (10) Extracellular matrix secretion cells such as Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other nonepithelial fibroblasts, Pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, and Stellate cell of perilymphatic space of ear; (11) Contractile cells such as Red skeletal muscle cell (slow), White skeletal muscle cell (fast), Intermediate skeletal muscle cell, nuclear bag cell of Muscle spindle, nuclear chain cell of Muscle spindle, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, and Red Blood Cell; (12) Blood and immune system cells such as Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, B cells, Natural killer cell, Reticulocyte, and Stem cells and committed progenitors for the blood and immune system (various types); (13) Sensory transducer cells such as Auditory inner hair cell of organ of Corti, Auditory outer hair cell of organ of Corti, Basal cell of olfactory epithelium (stem cell for olfactory neurons), Cold-sensitive primary sensory neurons, Heat-sensitive primary sensory neurons, Merkel cell of epidermis (touch sensor), Olfactory receptor neuron, Pain-sensitive primary sensory neurons (various types), Photoreceptor rod cell of eye, Photoreceptor blue-sensitive cone cell of eye, Photoreceptor green-sensitive cone cell of eye, Photoreceptor red-sensitive cone cell of eye, Proprioceptive primary sensory neurons (various types), Touch-sensitive primary sensory neurons (various types), Type I carotid body cell (blood pH sensor), Type II carotid body cell (blood pH sensor), Type I hair cell of vestibular apparatus of ear (acceleration and gravity), Type II hair cell of vestibular apparatus of ear (acceleration and gravity), and Type I taste bud cell; (14) Autonomic neuron cells such as Cholinergic neural cell (various types), Adrenergic neural cell (various types), Peptidergic neural cell (various types); (15) Sense organ and peripheral neuron supporting cells such as Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), and Enteric glial cell; (16) Central nervous system neurons and glial cells such as Astrocyte (various types), Neuron cells (large variety of types, still poorly classified), Oligodendrocyte, and Spindle neuron; (17) Lens cells such as Anterior lens epithelial cell, and Crystallin-containing lens fiber cell; (18) Pigment cells such as Melanocyte, and Retinal pigmented epithelial cell; (19) Germ cells such as Oogonium/Oocyte, Spermatid, Spermatocyte, Spermatogonium cell (stem cell for spermatocyte), and Spermatozoon; and Nurse cells such as Ovarian follicle cell, Sertoli cell (in testis), and Thymus epithelial cell, or mixtures of combinations thereof.

Pathogens suitable for use in infecting the TE models of this invention include, without limitation, any pathogen or microoranism known to cause diseases or other adverse responses in animals including humans. Exemplary examples include, without limitation, viruses such as the influenza viruses, autoimmunedeficiency viruese including the HIV viruses, or any other virus know to infect animals including humans, bacteria, prions, or any other biological pathogen. For a complete list of human and animal viruses the reader is referred to the following references of the world wide web virology.net/Big_Virology/BWirusList.html; or lancs.ac.uk/iss/a-virus/list.htm or other similar site, incorporated therein by reference.

Exemplary examples of pathogenic bacteria include, without limitation, Acinetobacter baumanii (Family Moraxellaceae), Actinobacillus spp. (Family Pasteurellaceae), Actinomycetes (actinomycetes, streptomycetes), Actinomyces, Actinomyces israelii, Actinomyces naeslundii, Actinomyces spp., Aeromonas spp. (Family Aeromonadaceae), Aeromonas hydrophila, Aeromonas veronii biovar sobria (Aeromonas sobria), Aeromonas caviae, Anaerobes, Non-Spore Forming, Gram-Positive Anaerobic Cocci, Peptostreptococcus spp., Streptococcus spp. (see separate listing below), Gram-Negative Anaerobic Cocci, Veillonella spp., Gram-Positive Anaerobic Bacilli, Actinomyces spp. (actinomycetes), Actinomyces israelii, Actinomyces naeslundii, Mobiluncus spp. (gram-positive cell wall, but stain gram-negative or variable), Propionibacterium acnes, Lactobacillus spp., Eubacterium spp., Bifidobacterium spp., Grarn-Negative Anaerobic Bacilli, Bacteroides spp. (see separate listing below), Prevotella spp., Porphyromonas spp., Fusobacterium spp., Bacillus spp. (Family Bacillaceae), Bacillus anthracis, Bacillus cereus, Bacillus subtilis, Bacillus thuringiensis, Bacillus stearothermophilus (used to test efficacy of autoclaves), Bacteroides spp. (Family Bacteroidaceae), Bacteroides fragilis (prototype endogenous anaerobic pathogen), Bordetella spp., Bordetella pertussis, Bordetella parapertussis, Bordetella bronchiseptica, Borrelia spp. (Order Spirochaetales; Family Spirochaetaceae), Borrelia recurrentis, Borrelia burgdorferi, Brucella spp., Brucella abortus, Brucella canis, Brucella melintensis, Brucella suis, Burkholderia spp. (formerly classified as Pseudomonas), Burkholderia pseudomallei, Burkholderia cepacia, Campylobacter spp., Campylobacter jejuni, Campylobacter coli, Campylobacter lari, Campylobacter fetus, Citrobacter spp. (Family Enterobacteriaceae), Clostridium spp., Clostridium perfringens, Clostridium difficile, Clostridium botulinum, Corynebacterium spp. (actinomycetes with mycolic acids, Family Corynebacteriaceae), Corynebacterium diphtheriae, Corynebacterium jeikeum, Corynebacterium urealyticum, Edwardsiella tarda (Family Enterobacteriaceae), Enterobacter spp. (Family Enterobacteriaceae), Family Enterobacteriaceae (clinically important enterics), Citrobacter, Citrobacter freundii, Citrobacter diversus, Enterobacter spp., Enterobacter aerogenes, Enterobacter agglomerans, Enterobacter cloacae, Escherichia coli, Opportunistic Escherichia coli, ETEC=enterotoxigenic E. coli, EIEC=enteroinvasive E. coli, EPEC=enteropathogenic E. coli, EHEC=enterohemorrhagic E. coli, EaggEC=enteroaggregative E. coli, UPEC=uropathogenic E. coli, Klebsiella spp., Klebsiella pneumoniae, Klebsiella oxytoca, Morganella morganii, Proteus spp., Proteus mirabilis, Proteus vulgaris, Providencia spp., Providencia alcalifaciens, Providencia rettgeri, Providencia stuartii, Salmonella spp., Salmonella enterica (proper nomenclature; encompasses all Salmonella; taxonomically only one species of Salmonella), Common nomenclature still in use: Salmonella typhi, Salmonella paratyphi, Salmonella enteritidis, Salmonella cholerasuis, Salmonella typhimurium, Serratia spp., Serratia marcesans, Serratia liquifaciens, Shigella spp., Shigella dysenteriae, Shigella flexneri, Shigella boydii, Shigella sonnei, Yersinia spp., Yersinia enterocolitica, Yersinia pestis, Yersinia pseudotuberculosis, Enterococcus spp. (Lancefield Group D specific carbohydrate) (gamma hemolytic, occasionally alpha or beta) (formerly classified as Group D streptococci), Enterococcus faecalis, Enterococcus faecium, Erysipelothrix rhusopathiae, Escherichia coli (Family Enterobacteriaceae), Opportunistic Escherichia coli, ETEC=enterotoxigenic E. coli, EIEC=enteroinvasive E. coli, EPEC=enteropathogenic E. coli, EHEC=enterohemorrhagic E. coli, EaggEC=enteroaggregative E. coli, UPEC=uropathogenic E. coli, Francisella tularensis, Haemophilus spp. (Family Pasteurellaceae), Haemophilus influenzae, Haemophilus ducreyi, Haemophilus aegyptius, Haemophilus parainfluenzae, Haemophilus haemolyticus, Haemophilus parahaemolyticus, Helicobacter spp., Helicobacter pylori, Helicobacter cinaedi, Helicobacter fennelliae, Klebsiella pneumoniae (Family Enterobacteriaceae), Legionella pneumophila, Leptospira interrogans (Order Spirochaetales; Family Leptospiraceae), Serogroups: canicola, pomona, icterohaemorrhagiae, Listeria monocytogenes, Micrococcus spp. (Family Micrococcaceae), Moraxella catarrhalis (taxonomic confusion) (Family Moraxellaceae or Family Neisseriaceae), Formerly classified as Neisseria, More recently classified as Branhamella, Morganella spp. (Family Enterobacteriaceae), Mycobacterium spp. (actinomycetes with mycolic acids, Family Mycobacteriaceae), Mycobacterium leprae, Mycobacterium tuberculosis, Nocardia spp. (actinomycetes with mycolic acids, Family Nocardiaceae), Nocardia asteroides, Nocardia brasiliensis, Neisseria spp. (Family Neisseriaceae), Neisseria gonorrhoeae, Neisseria meningitidis, Pasteurella multocida (Family Pasteurellaceae), Plesiomonas shigelloides (Family Plesiomonadaceae), Propionibacterium acnes, Proteus spp. (Family Enterobacteriaceae), Proteus vulgaris, Proteus mirabilis, Providencia spp. (Family Enterobacteriaceae), Pseudomonas aeruginosa (Family Pseudomonadaceae), Rhodococcus spp. (actinomycetes with mycolic acids, Family Nocardiaceae), Salmonella spp. (Family Enterobacteriaceae), Salmonella enterica (proper nomenclature; encompasses all Salmonella; taxonomically only one species of Salmonella), Common nomenclature still in use: Salmonella typhi, Salmonella paratyphi, Salmonella enteritidis, Salmonella cholerasuis, Salmonella typhimurium, Serratia marcescens (Family Enterobacteriaceae), Shigella spp. (Family Enterobacteriaceae), Shigella dysenteriae, Shigella flexneri, Shigella boydii, Shigella sonnei, Staphylococcus spp. (Family Micrococcaceae) (catalase positive), Staphylococcus aureus (coagulase-positive), Staphylococcus epidermidis(coagulase-negative), Staphylococcus saprophyticus (coagulase-negative), Stenotrophomonas maltophilia, Originally classified as Pseudomonas, More recently classified as Xanthomonas, Streptococcus pneumoniae (no group specific carbohydrate) (alpha hemolytic), Streptococcus spp. (FAMILY Streptococcaceae) (catalase negative), Group A streptococci (beta hemolytic), Streptococcus pyogenes, Group B streptococci (beta hemolytic, occasionally alpha or gamma), Streptococcus agalactiae, Group C streptococci (beta hemolytic, occasionally alpha or gamma), Streptococcus anginosus, Streptococcus equismilis, Group D streptococci (alpha or gamma hemolytic, occasionally beta), Streptococcus bovis, Group F streptococci (beta hemolytic), Streptococcus anginosus, Group G streptococci (beta hemolytic), Streptococcus anginosus, Viridans streptococci (no group specific carbohydrate) (alpha or gamma hemolytic), Streptococcus mutans, Streptococcus salivarius group, Streptococcus sanguis group, Streptococcus mitis group, Streptococcus milleri group, Streptomyces spp. (actinomycetes, streptomycetes), Treponema spp. (Order Spirochaetales; Family Spirochaetaceae), Treponema pallidum ssp. pallidum, Treponema pallidum ssp. endemicum, Treponema pallidum ssp. pertenue, Treponema carateum, Vibrio spp. (Family Vibrionaceae), Vibrio cholerae Serogroups O1 and O139 that produce specific cholera enterotoxin are responsible for classic cholera epidemics, Vibrio cholerae O1 (Serogroup O1) Biotypes: cholerae (classical), el tor, Biotypes are further subdivided into serotypes: ogawa, inaba, hinkojima, Vibrio cholerae O139 (Serogroup O139) Newly recognized in 1992 (classic case of an important emerging pathogen), Non-agglutinable vibrios (NAGs) or non-cholera vibrios (NCVs) Identical to Vibrio cholerae O1, but do not agglutinate in O1 antiserum, Vibrio parahaemolyticus, Vibrio vulnificus, Vibrio alginolyticus, Vibrio mimicus, Vibrio hollisae, Vibrio fluvialis, Vibrio metchnikovii, Vibrio damsela, Vibrio furnisii, Yersinia spp. (Family Enterobacteriaceae), Yersinia enterocolitica, Yersinia pestis, Yersinia pseudotuberculosis, or mixtures or combinations thereof.

### Lung Tissue Experimental Section of the Invention

### Isolation of Somatic Lung Progenitor Cells (SLPCs)

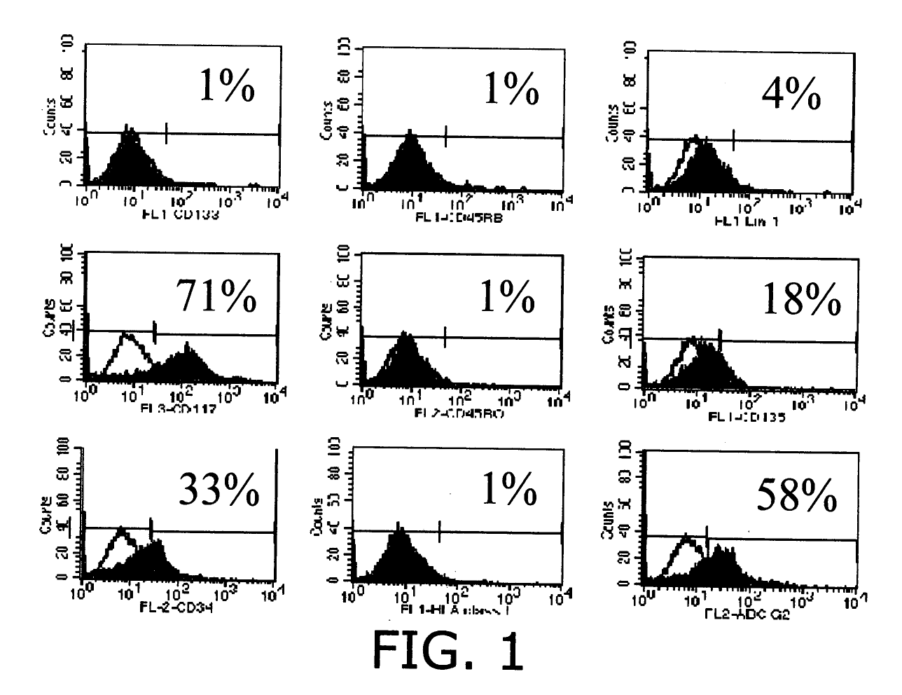

SLPCs were isolated from a number of mammalian (murine, ovine, human) sources. Discarded lung tissue from two idiopathic pulmonary fibrosis patients undergoing lung transplants as well as one surgical pathology specimen were used as a source for human SLPCs. Cells were isolated as described herein. Referring now to FIG. 1 {1}, human and murine SLPCs (data not shown) were negative for CD45 and MHC class I and highly positive for CD34, CD117 (c-kit), CD135 (fms-like tyrosine kinase-3 (flt-3). Murine SLPCs were also positive for stem cell antigen-1 (Sca-1) (99%) (data not shown).

### In Vitro Differentiation and Development of Tissue

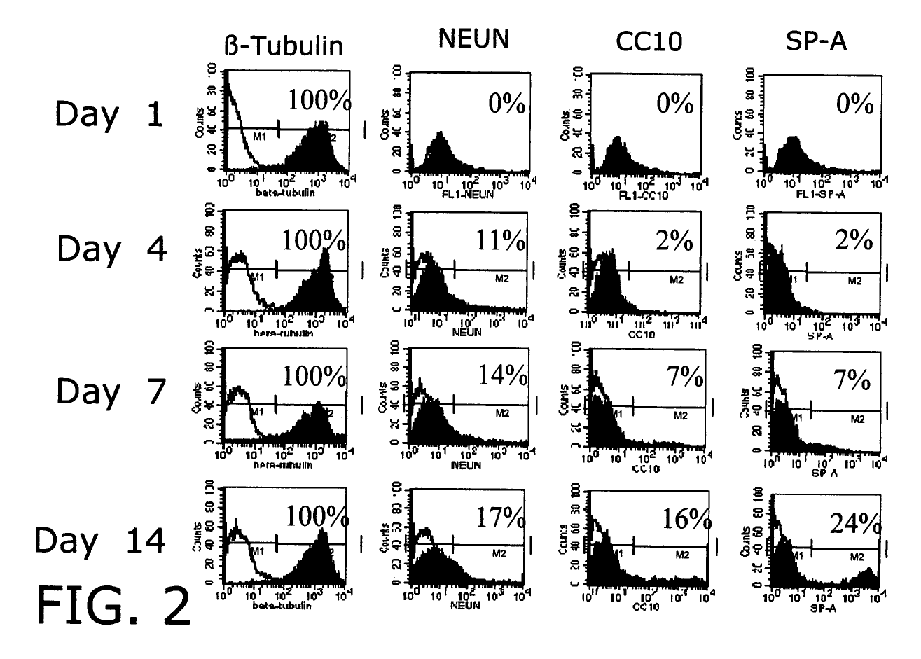

To determine whether SLPCs generate multiple cell types, isolated cells were cultured in the presence of growth factors (epidermal growth factor [EGF] and fibroblast growth factor [FGF]) as well as 10% fetal calf serum and 20% bone marrow stromal cell derived cell free conditioned culture media, for 0, 4, 7 and 14 days. Evaluation of CC10, a lung marker used to study the development of Clara cells in fetal lung, of SP-A and of neuron-specific enolase (NEUN) used as a general neuroendocrine marker showed that, immediately after isolation, the selected cell population did not express these markers of characteristic of differentiated lung cells as shown in FIG. 2 {2}. Flow cytometric analysis of markers of mature lung evaluated at days 1, 4, 7, and 14 of culture showed that there was no production of CC10, SP-A or NEUN, on day 1 of isolation or prior to day 4 of culture (data not shown), but that by days 4, 7, and 14 of culture increasing numbers of positive cells were seen as the time in culture increased (see FIG. 2). Flow cytometric evaluation of SLPCs at all stages of differentiation on all days of culture were shown to be positive for the control β-tubulin as were fully differentiated cultures of mature lung cells (see FIG. 2).

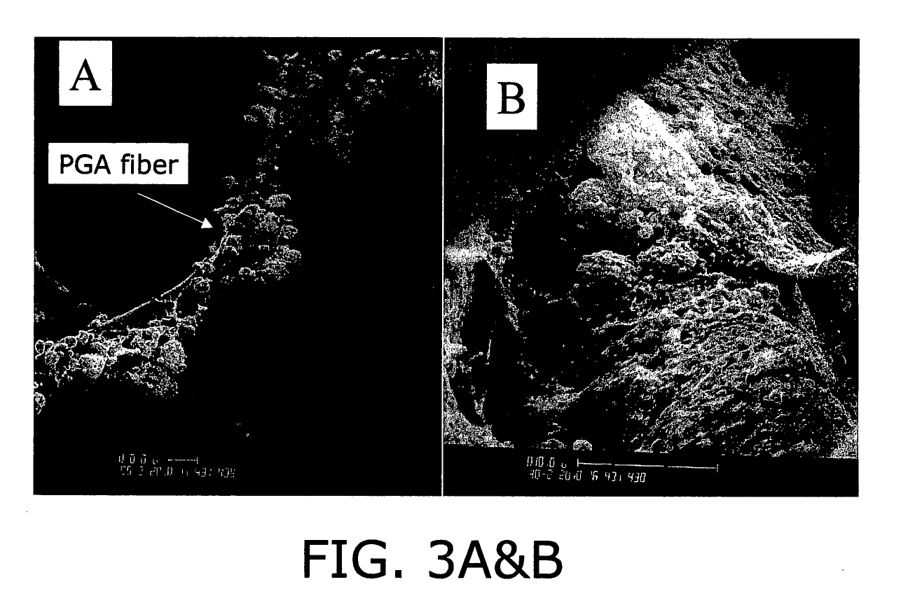

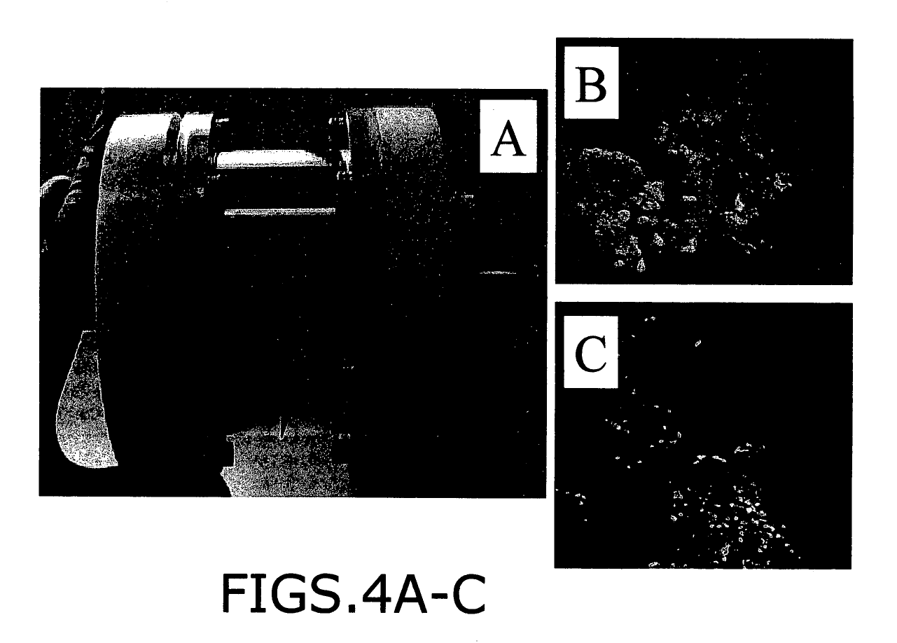

Referring now to FIGS. 3A&B, scanning electron microscopy micrographs of lung progenitor cells growing in vitro on the polyglycolic acid (PGA) scaffold are shown. The cells maintained in flasks containing the synthetic polymer scaffold began to attach to the PGA fibers on day 1 and continued to develop, differentiate and produce extracellular matrix material through 8 weeks of culture. The data evidences the formation of in vitro mammalian (murine or human) tissue engineered lung that consistently produce discrete pieces of engineered lung which we refer to as “organoids” (see FIG. 4A). The results of two 4 week cultures using murine (see FIGS. 4A&B) or human (see FIG. 4C) SLPCs as the progenitor population cell source are seen in FIGS. 4A-C. We are now able to produce 0.5-2 grams of TE lung from a starting population of 1−2×10⁵ cells by 8 weeks of culture. As can be seen the large oval tissue engineered organoids (see arrow in FIG. 4A) are well formed by 4 weeks. Measurable levels of pro-surfactant protein C (pro-SPC), the intracellular non secreted form of surfactant protein C, is produced by both murine (see FIG. 4B, pro-SPC is green) as well as human TE lung (see FIG. 4C) cultures by 4 weeks indicating that type II pneumocytes are present in-the TE tissue organoids.

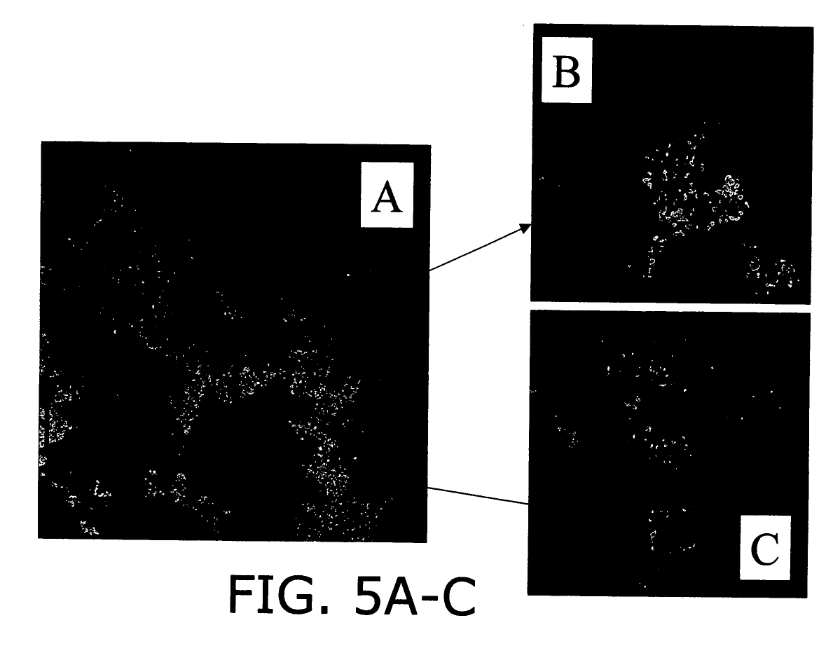

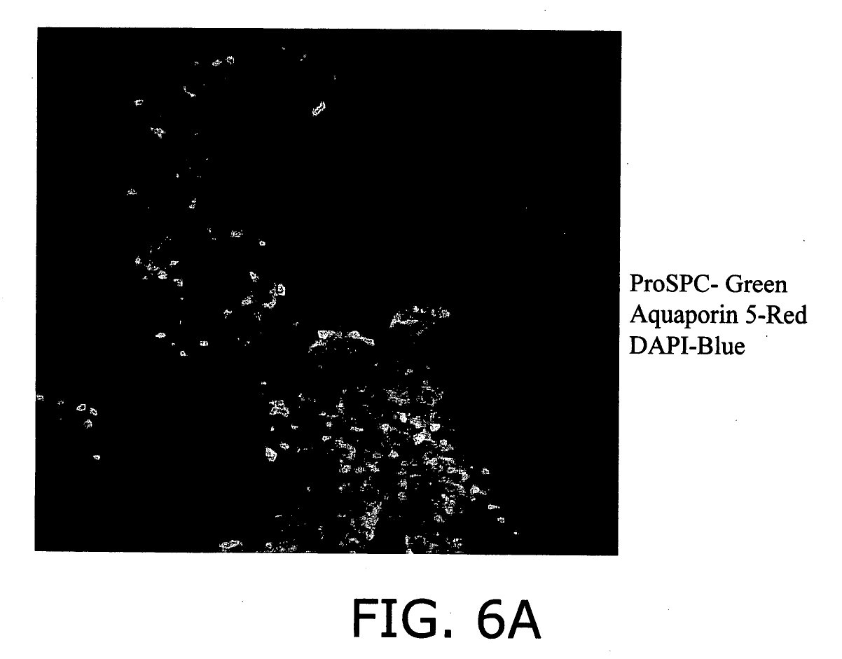

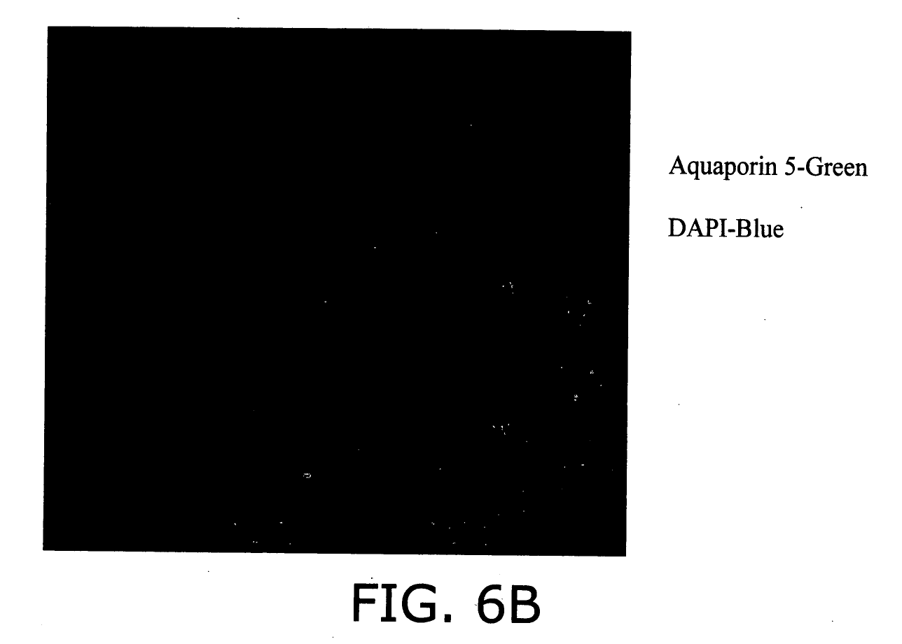

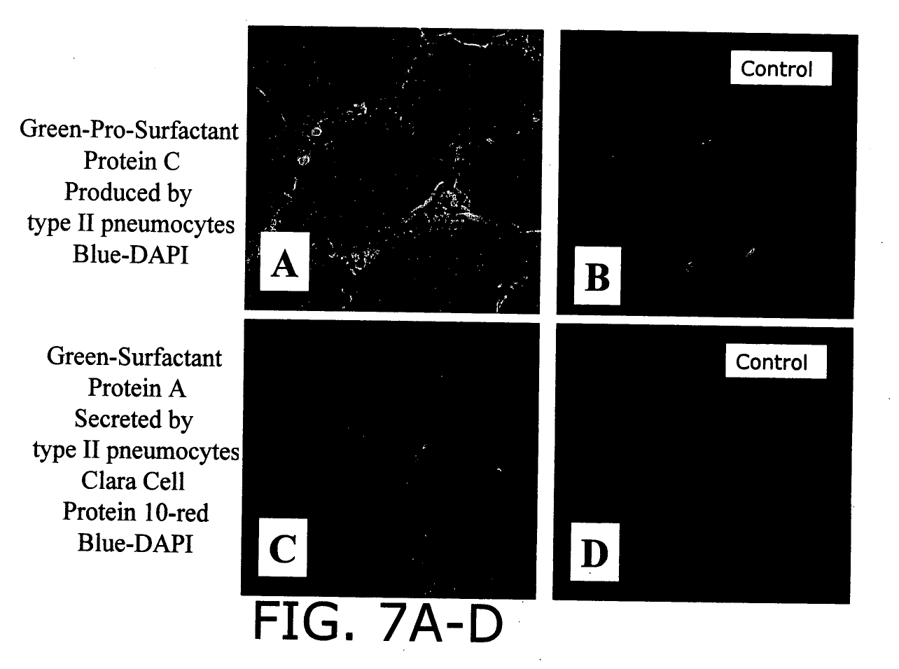

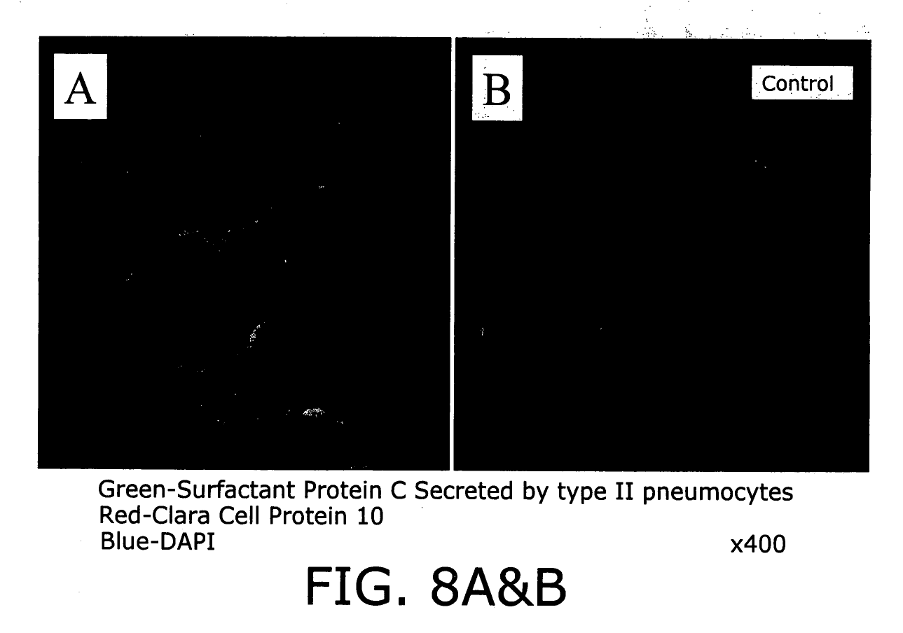

Immunohistochemical analysis of frozen sections of the normal human lung shows expression of surfactant protein C(SPC) which is the secreted form of this protein (FIG. 5C green is SPC, red is a pan leukocyte marker CD45). The nuclear stain DAPI which appears blue was used to label the cell nucleus (see FIGS. 5A-C). Aquaporin-5 (AQ-5) staining of thick sections (10-11 μm) of TE human lung demonstrate the presence of type I pneumocytes (see FIG. 6A: red is AQ-5, Green is Pro SPC and FIG. 6B: green is AQ-5 alone). Frozen sections show that pro SPC (see FIG. 7A and control FIG. 7B), surfactant protein A (secreted form, see FIG. 7C and control FIG. 7D) and Clara cell protein 10 (see FIG. 7C and control FIG. 7D) are produced in murine TE lung cultures. The same is true in human TE lung cultures where pro SPC (see FIG. 8A and control FIG. 7B), surfactant protein A (secreted form, data not shown) and Clara cell protein 10 (see FIG. 8B and control FIG. 7B) are produced. Clara cells and type II pneumocytes are present in both murine and human TE lung cultures as early as 3 weeks of culture (data not shown).

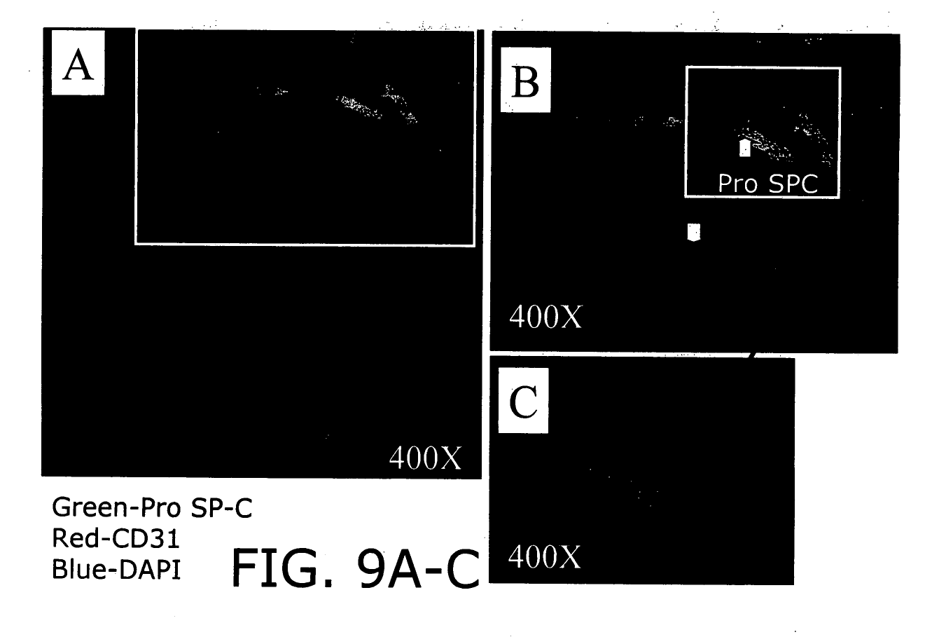

In the developing TE organoids by 6-8 weeks, we demonstrated some expression of CD31 an endothelial cell marker in close proximity to pro-SPC (type II pneumocytes) positive cells in human (see FIGS. 9A-C) and murine (data not shown) cultures. The presence of endothelial cells in the TE lung systems is significant because endothelial-1 (ET-1), secreted by endothelial cells, plays a key role in the stimulation of surfactant secretion by alveolar type II cells. Elements of the lung proteome such as endothelian-1 (ET-1), a potent regulator of smooth muscle tone and inflammation, also may play a key role in diseases of the airways, pulmonary circulation, and inflammatory lung diseases both acute and chronic. The system of this invention can then be used to study the inter-relationship between influenza exposure and ET-1 production followed by surfactant secretion.

Currently, we are adjusting the growth factor and cytokine cocktails to enhance development of both CD31 positive endothelial cells and Type I pneumocytes from the SLPC population. In vivo studies, where SLPC from ovine, murine or human sources, were grafted onto the back of a nude mouse support the potential for the SLPC population to produce the development of neuroendoctrine cells, Clara cells, type I and II pneumocytes, endothelial cells, goblet cells and smooth muscle (data not shown).

### Validation of the Model

One of the inventors previously concentrated on the role of leukocytes in the host response to influenza exposure. She demonstrated that leukocytes can become infected with influenza, although the infection is abortive in these cells and that apoptosis is triggered in infected cells through a Fas-FasL pathway.

The present in vitro bronchiole-alveolar model are designed so that: (1) the model structurally and architecturally looks like the bronchiole-alveolar junction, (2) the cell types that are found in this region in native lung are also found in the model in approximately the correct proportions, (3) proteins produced in native lung are also produced in the model, (4) microbiologically the model functions like normal lung and finally, and (5) pharmacologically the model responds the same way that normal lung does.

To evaluate whether the TE models responds to microbial exposure, equal weights of TE material were sham exposed or exposed to infectious virus. In order to estimate the amount of virus to use in the TE cultures cells from lung cell lines were counted and pelleted by centrifugation. The pellets were then weighed and compared to the total weight of the TE lung organoids. This allowed for a rough approximation of the possible cell number in the TE organoid. For human TE lung exposures, a clinical isolate A/Marton/43 (H1N1) was used. Other strains of virus including A/Udorn (H3N2) as well as avian-human reassortant strains have been used in this system with excellent results. For studies of TE murine lung, we have used mouse adapted PR/34 (H1N1).

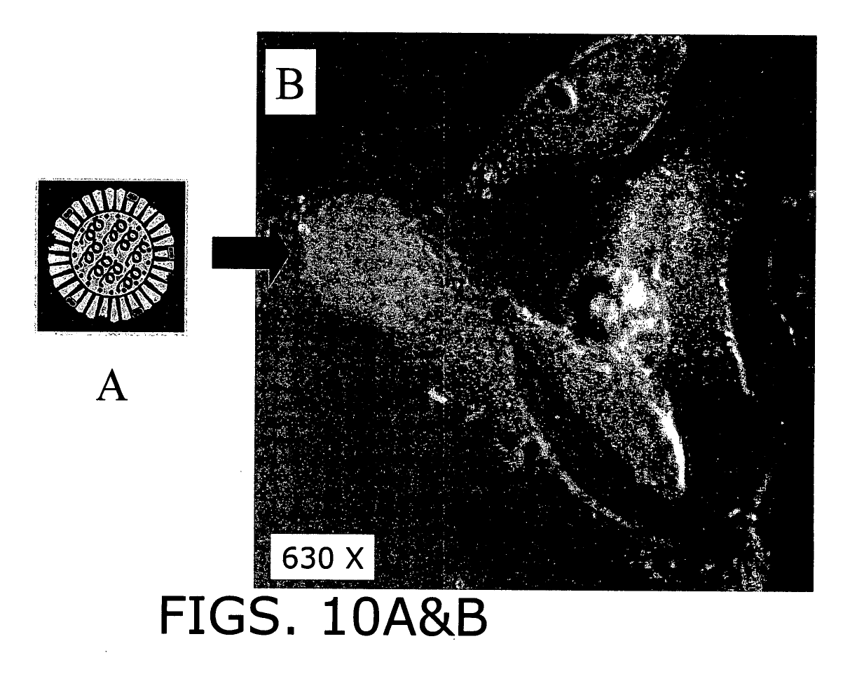

In some virus-exposures, in order to examine cellular uptake of virus, influenza A virus A/Marton/43 (H1N1), was labeled with fluorescein isothiocyanate (FITC) as previously described which places a number of FITC molecules on surface viral hemagglutinin (H) and neuraminidase (NA) (see the diagram in FIG. 10A). Tests of the in vitro TE human model demonstrated that there is uptake of FITC-labeled virus into cellular endosomes (see FIG. 10B) with quenching of the endosomaly transported virus after lysosomal-endosomal fusion. The data also showed that the kinetics of virus uptake, endosomal-lysosomal fusion (as measured by quenching of FITC), production of viral proteins and budding of new virions occurs in a pattern similar to that for cell line in vitro virus infection/culture.

To examine the infection of cells, TE human organoids were cultured on glass coverslips for 2 hours after virus exposure or were allowed to continue in the bioreactor chamber until the organoid was frozen and sectioned. Immunocytochemical staining was performed to monitor the production of specific viral proteins using antibodies specific for H1, N1, matrix (M) or nucleoprotein (NP) or to determine cell infection using a mixture of anti-H, -NA and -M antibodies followed by either a FITC (green) or rhodamine (red) tagged anti-murine secondary antibody.

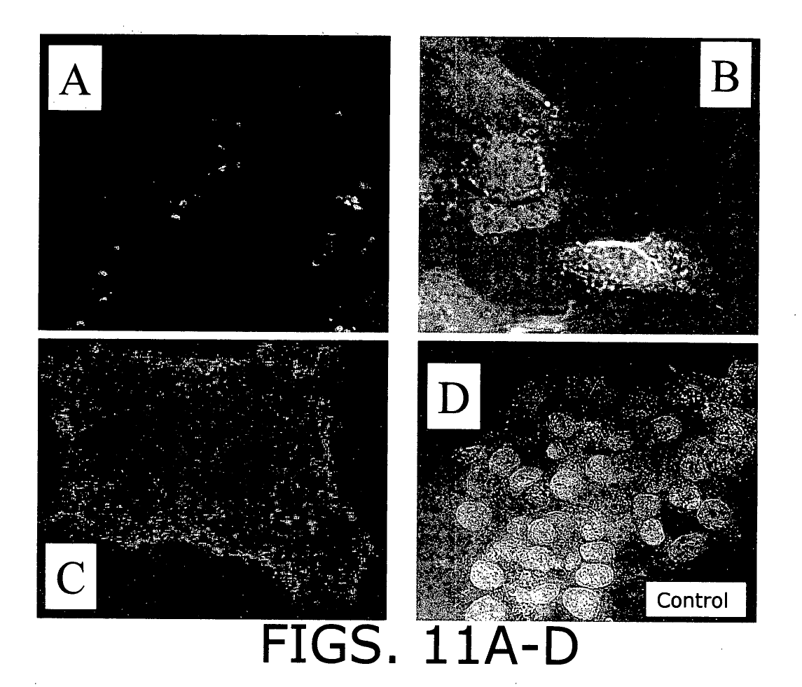

In thick sections (10-11 μm), infected cells were seen in discrete patches through out the entire organoid within 2 hours after virus exposure (see FIG. 11A, infected cells-green, DAPI nuclear stain-blue). These patches spread over time and within 24 hours large areas of virus infected cells are seen (data not shown). Production of virus by individual cells in the organoid show normal patterns of budding as shown by the staining of H and NA as virus was released from individual cells in the organoid (FIG. 11B and FIG. 11C, H and NA-green, DAPI-blue). Staining of a sham exposed TE organoid using the same antibody combination as in FIG. 11B and FIG. 11C is shown in FIG. 11D. There was little non-specific staining of the sham exposed culture as expected.

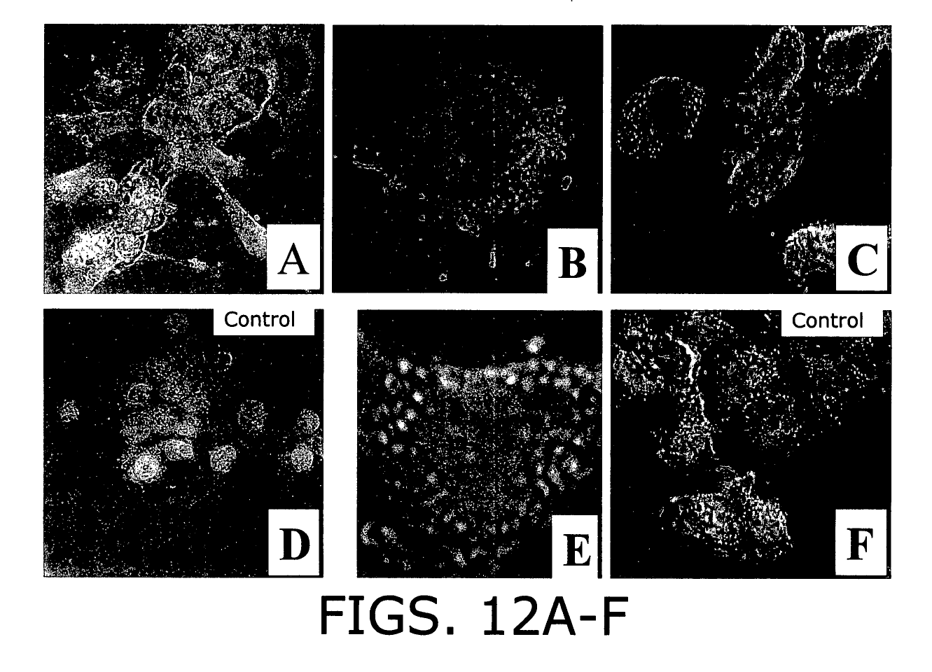

To measure the pharmacological respond of the TE model in a manner similar to native lung tissue, we treated TE cultures with a neuraminidase (NA) inhibitor. NA inhibitors are class of anti-influenza drugs used for both prophylaxis and treatment of influenza virus infections. The drugs are highly potent sialic acid (SA) analogues that selectively target the NA enzyme of both influenza A and B viruses. The viruses interact with NA with a higher affinity than SA in a slow-binding manner, thereby preventing the cleavage of SA molecules from host cell receptors required for viral release. Pharmacologically, the TE organoid reacts in a manner similar to 2 dimensional cell line or single cell culture and agents that inhibit virus release such as NA inhibitor (see FIGS. 12A-C, virus is stained green, sham exposed culture stained with the same mixture of ant-H, -N, and -M in 11D) or other antivirals known to block virus budding (antiviral kindly provided by Functional Genetics, Rockville Md.) (see FIG. 12E, virus is stained red, sham exposed culture stained with the same mixture of ant-H, -NA, and -M antibodies in FIG. 12F). The accumulation of clumped virus in the NA treated cultures is shown in FIGS. 12A-C. In cultures treated with the antiviral that blocks budding of newly formed virus, an accumulation of virus just below the surface of the cell membrane is shown in FIG. 12F.

The average virus titers for three experiments using cells from 3 different lung tissue donors in 4 week versus 8 week TE organoid cultures with and without addition of the same NA inhibitor or the antiviral provided by Functional Genetics, Rockville, Md.) is shown in Table 1 below. The sialidase (nNA) inhibitor 4-guanidino-2,4-dideoxy-2,3-dehydro-N-acetylneuraminic acid (4-guanidino-Neu5Ac2en) was used in these studies at a concentration of 5 microM as was the anti-budding antiviral provided by Functional Genetics.

TABLE 1
Engineered Tissue Response to Antiviral Agents

| | | Neuraminidase | Other Antiviral |
|---|---|---|---|
| Sample | No Treatment | inhibitor | Treatment |
| 4 week TE | 7.4 × 10⁸ | 4.93 × 10⁵ | 2 × 10⁶ |
| organoid culture | | | |
| 8 week TE | 5 × 10⁷ | 2.7 × 10⁵ | 8.3 × 10⁵ |
| organoid culture | | | |

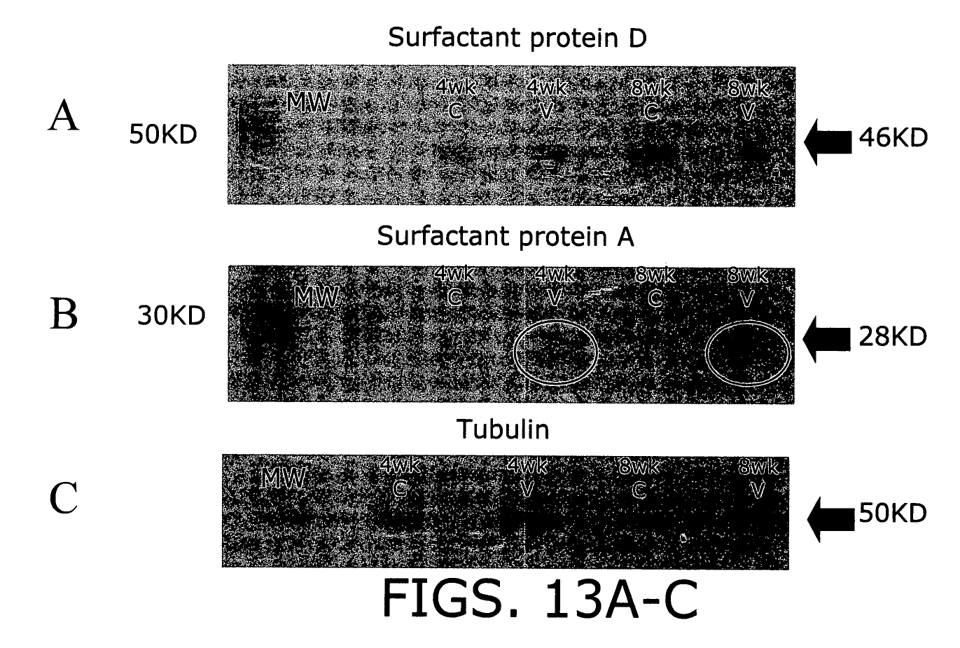

Collectins are secreted collagen-like lectins that bind, agglutinate, and neutralize influenza A virus (IAV) in vitro. Surfactant proteins A and D (SP-A and SP-D) are collectins expressed in the airway and alveolar epithelium and may play a role in the regulation of IAV infection. Because the model is designed to measure the innate response of the cells that comprise the bronchiole-alveolar junction, we examined the influence of virus exposure on surfactant protein A and D production in the TE model. Here we demonstrated that surfactant was being secreted by cells that comprise the TE model and we determined whether virus exposure increased the production of these components of lung surfactant. Same weight sham and virus exposed cultures were incubated for 24 hours after exposure and then the cultures were harvested and cell lysates were made. Immunoprecipitations of surfactant protein A, D and control β-tubulin were done on lysates from sham and virus-exposed TE cultures. Levels of surfactant protein D remained constant in both virus and sham exposed samples of tissue from 4 and 8 week cultures of TE lung. Levels of surfactant protein A varied and sham exposed cultures produced low levels of this protein compared to the levels seen in virus exposed cultures (either 4 or 8 week see FIGS. 13A-C). This finding is important validation data because SP-A has been shown to act as an opsonin in the phagocytosis of other viruses by alveolar macrophages and amounts of SP-A have been shown to vary with virus exposure.

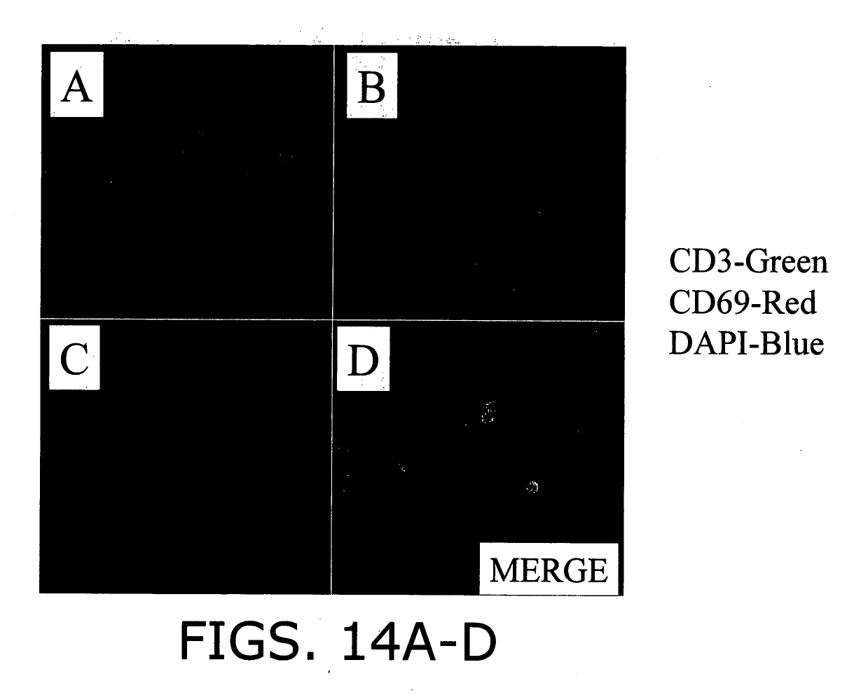

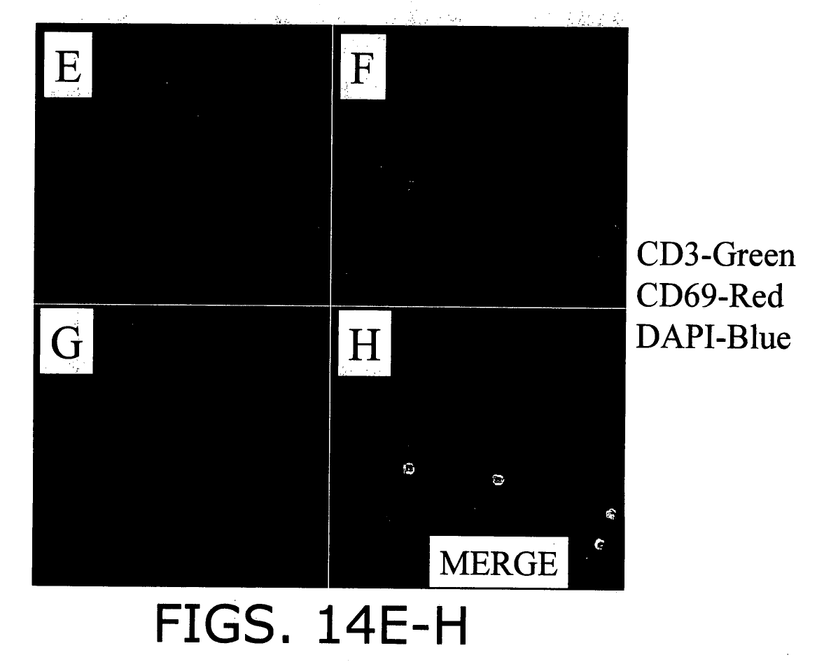

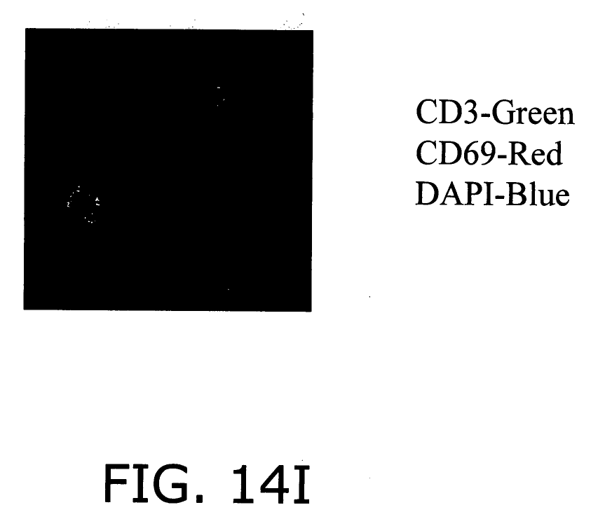

To further validate the TE model, we combined immune cells with the engineered tissues using either human or murine lung. This is quite easy in the case of the murine TE lung where we added same strain (BALBc) leucocytes to the engineered tissue at specific stages of development. In the case of human tissues, the preparation of such a combined system was a bit more complex and we used half-matched (Human leukocyte antigen, HLA haplomatched) leucocytes. Use of side by side TE cultures with and without the addition of leucocytes will make for an easier comparison between native and TE lung tissue. We have had excellent results in developing the murine TE immune cell cultures due to the ease in obtaining same strain murine cells. In FIGS. 14A-I, we show results of a human HLA-haplo-matched immune cell/TE lung culture. Sections of lung were stained with a pan t-lymphocyte marker CD3 (in green) and a marker of cell activation, CD69 (in red). In TE-leukocyte cocultures, with sham primed leucocytes (see FIGS. 14A-D) there is no specific response to influenza A by the lung associated immune cells. In cultures using leucocytes primed in vitro with heat killed influenza A/Marton/43 we see that a small portion of the T-lymphocytes are activated by exposure to live virus (see FIGS. 14E-H). In both the sham (see FIG. 14D) and virus exposed (see FIG. 14H) cultures, the merging of the red and green staining is shown. DAPI was used to stain the nuclei in both preparations. In FIGS. 14D-H representing the merged image (red-CD69, green-CD3), it is apparent that primed-leucocytes other than CD3+ T-lymphocytes are activated by exposure to the virus.

### Design and Methods

Discarded human tissue was collected using a University of Texas Medical Branch IRB approved protocol. In brief, tissues from both sexes and all age ranges are accepted for use in this study. Human tissues were not used from autopsy or surgical pathology cases after diagnosis of infectious disease or cancer.

### Criteria for Determining the Successful Generation of the TE Lung Model

The in vitro tissue model are designed to meet the following 3 basic criteria: (1) the culture system must be proliferative and self sustaining in culture within the rotary bioreactor for at least-8 weeks; (2) the TE model must possess a three-dimensional architecture on histologic sectioning and examination consistent with the bronchiole-alveolar junction. We intend to examine stained microscopic sections of the TE organoids and compare structural features and characteristics with native lung (murine or human) architecture at the site of the bronchiole-alveolar junction; and (3) the TE model must express specific mature lung cell antigens/products that mirror the expression pattern and distribution of lung epithelium in vivo.

Expression of protein products was evaluated by immunocytochemistry and the results are reviewed and compared to normal lung (murine, human). Cell types to be examined include, without limitation, Type I and Type II pneumocytes, Clara cells, endothelial cells, neuroendocrine cells, smooth muscle and mucin secreting cells or goblet cells. Cell types and products are evaluated using immunohistochemical staining or staining with cell type specific lectins with analysis by confocal microscopy. Cell products are examined by western blotting or through the immunoprecipitation of cell products from cell lysates followed by gel electrophoresis. A list of some of the products examined is tabulated in Table 2.

TABLE 2
Engineered Tissue Protein and Marker Productions

| Cell type | Protein Products | Markers |
|---|---|---|
| Type I pneumocyte | Aquaporin 5 | Lycopersion esculentum, |
| | | Ricinus communis binding, ICAM |
| Type II pneumocyte | Surfactant Protein A, B, C, D, as well as secretion | ICAM, VCAM, CD44, |
| | of MIP-1 alpha and RANTES after TNF | Maclura prolifera binding |
| | stimulation, cytokeratin 19 | |
| Clara cell | CC10, CC16, Surfactant Protein B, anti- | |
| | leukoproteases inhibitor | |
| Neuroendocrine cell | Neuroenolase, serotonin, leu 7 NK cell | N-CAM |
| | antigen | |
| Smooth Muscle | Alpha-actin, Tropoelastin, | |
| Mucin producing or | Apomucin or mucin 2, 4, 5 | |
| goblet cell | | |
| Endothelial cells | Endothelien 1 | CD31, PECAM |
| Fibroblasts | Interleukin 6, Tropoelastin, | Il-4 receptor |

Relatively little is known about stem and progenitor cells that exist in the lung or the process of their differentiation and organization into lung tissue. As has been presented in some detail in above, we demonstrated that these precursor cells can differentiate into numerous cell types that produce Clara cell protein 10 (CC 10), neuron-specific enolase, cytokeratin, and surfactant proteins A and C(SP-A or SP-C) prior to formation of in vitro cell/polymer constructs. Thus, the SLPC population is (1) a mixture of multipotent somatic precursor cells capable of differentiating into progeny with multiple differentiation phenotypes, and/or (2) mixtures of unipotent somatic progenitor cells, each giving rise to an array of lung specific single-cell lineages. Our data are based on the selected differentiation and maturation of a heterogeneous population of unipotent or multipotent stem cells isolated from excised lung, which develop into tissues that include the bronchiole-alveolar junction, alveolar areas and small bronchioles.

The present invention is also related to the determination of culturing and bio-reactor conditions sufficient to produce and support long-term, stable culture of engineered tissue that structurally and functionally resembles lung epithelium or other tissues such as lymphatic tissue. The present invention also relates to the development of optimal conditions for infection of murine and human Tissue engineered (TE) models with influenza A virus or other pathogens or toxins or mixtures or combinations thereof. Murine TE lung tissues are developed along side of human TE lung in order to enhance validation of the influenza pathogenesis model system and to provide additional pathogenesis data in other mammal and other species as well. We have and continue to determine cell types and cell products expressed in human and murine TE lung compared to normal lung tissues. Our evaluation of the lung proteome and the dynamic collection of specialized lung proteins, includes, without limitation, evaluation of Clara cell, neuroendocrine cell and pneumocyte (Types I and II) products for both murine and human, native and TE lung.

TE lung organoids are grown in triplicate culture from 3 murine donors or in triplicate culture from 3 human tissue donors. TE tissues are weighed and each member of a triplicate culture are grown to the same or similar weight of tissue. Murine tissue are obtained for development of the TE organoids by humane sacrifice of 2 BALBc male mice following IACUC guidelines. Human tissue are obtained from discarded human pathology or cadaver tissues following University of Texas Medical Branch IRB guidelines. The isolation and characterization of progenitor populations used are described in the method sections below. Triplicate cultures of murine or human TE organoids are evaluated for cell types produced, and protein products generated, in order to determine variability between side-by-side cultures. Normal murine or human lung are compared to TE lung from both 4 week and 8 week old TE cultures.

### Collection of Somatic Lung Progenitor Cells (SLPCs)

Murine pulmonary cells are obtained from the lungs of male BALBc mouse (Charles River Laboratories, Wilmington, Va.), essentially following the known protocol. Human lung tissues will be obtained from lungs of patients undergoing lobotomy. The participants must all have normal lung function, use no medication, and not be infected or have had lung tissue removed due to cancer. Normal parts of the tissue are washed with PBS, and pleural tissue and bronchi are removed after resection.

Briefly, lungs are surgically removed, rinsed in Hanks Balanced Salt Solution (HBSS; Cellgro, Herndon, Va.). The pleura are then removed and the tissue is minced, triturated, and digested with 0.5% trypsin in PBS for 5 and 20 min, respectively. Following quenching of the trypsin with Dulbecco's modified Eagle medium containing 10% FBS (Hyclone, Logan, Utah) and filtration through a 70 μm filter (BD Falcon, San Jose, Calif.), the cell suspension is pelleted for 5 min at 800 rpm. The pellet are resuspended for 30 s in distilled water to remove red blood cells by hypotonic lysis, followed by the addition of PBS (Cellgro). The cells are then washed once more in Ca₂/Mg₂ containing PBS, resuspended in complete medium (DMEM+10% fetal bovine serum+antibiotics) and counted. Cell viability is assessed in a fluorescent microscope using the live/dead assay (Molecular Probes, Eugene, Oreg.), according to the manufacturer's instructions.

For the initial 24 hours, primary isolates are cultured in DMEM medium (Cambrex), supplemented with 10% fetal bovine serum (Hyclone, Logan, Utah), L-glutamine, penicillin-streptomycin antibiotics, and 1% insulin transferrin-selenium (ITS) supplement containing linoleic acid and BSA (BD Biosciences, San Jose, Calif.).

After 24 hours, the culture media are switched to one of two serum free media formulations: serum-free DMEM with the same supplements noted above (SF-ITS) was used in early experiments, whereas in later series of experiments, a serum free, tissue-specific, growth factor-defined medium (SFGF) containing FGF-7 (12.5 ng/mL), FGF-10 (25 ng/mL), and bFGF (12.5 ng/mL) in an 10:1 mixture of DMEM:F12 with L-glutamine and penicillin-streptomycin antibiotics was used to enhance epithelial cell differentiation and tissue construct morphogenesis.

All cell culture are carried out at 37° C. in a 5% CO₂ humidified incubator. Somatic lung progenitor cells are then isolated based on cell size and cell density using counter current centrifugal elutriation in order to isolate the small (5-7 μm) progenitor population as using a method previously described for isolation of small lymphocytes.

### Seeding and Culture of Lung Progenitor Cells and Matrix in the Bioreactor

The general concept of using tissue-engineering techniques to grow tissues has dramatically expanded within the last ten years. Recently, growing bone and cartilage structures has been the primary focus of most tissue-engineering initiatives. Current advances in the development of different matrices for cell attachment has progressed from naturally occurring polymers to synthetic polymers. Work in this area has improved cell attachment techniques and resulted in strengthening the resulting tissue. By incorporating simple engineering and polymer science techniques that maintain a three-dimensional structure, cells can attach to the polymer and secrete their matrix while maintaining the shape desired. In other words by maintaining the cells in a three dimensional orientation during growth and development, these polymers act to support cellular function by guiding the spatially and temporally complex multicellular processes of tissue formation and regeneration, thereby creating appropriately configured tissue constructs with similar morphological characteristics as the native tissue.

Although two-dimensional in vitro assays are still applied in many cell culture studies, there is increasing agreement that three-dimensional matrices provide better model systems for physiologic situations. One such method is to study the generation of differentiated tissue in microgravity via an in vitro 3 dimensional cell culture system known as a bioreactor. Bioreactor technology has made it possible to decrease cell stress and create tissues that are stronger and capable of maintaining their shapes in vitro. Current bioreactors use a constant flow of media and oxygen to bathe the cells simulating a normal in vivo environment thereby avoiding the necrosis seen in long-term in vitro cell polymer studies. This technology offers the investigator the ability to grow tissues of a particular organ in vitro for biochemical, injury or pathogen-exposure studies and potentially for in vivo implantation. Investigators are then be able to observe first hand how bioreactor-grown lung cells respond to different injuries, toxins, pathogenic organisms and therapies. We used a combination of Polyglycolic Acid (PGA) fibers with a 30% mixture of a hydrogel, pluronic F-127 to encourage cell PGA interaction and support of its three dimensional orientation by rotating the cells within a rotary bioreactor where the cells will use the PGA fibers as an template.

Formation of the cell/polymer (PGA) constructs was accomplished as set forth below. A mixed population of SLPCs is seeded onto PGA and cultured in an incubator for three days prior to placing the cell/PGA constructs into a rotary bioreactor (Synthecon, Houston, Tex.) in either a 10 mL or 100 mL chamber. The cell-polymer construct (1 cm) is placed inside of a rotating culture vessel allowing the external media (100 mL) to bathe the tissue. The 1 cm diameter construct is maintained in suspension by balancing the sedimentation induced by gravity with centrifugation caused by vessel rotation. The initial rotational speed is adjusted so that the cell-polymer construct will rotate synchronously with the vessel, so that there is a low shear force placed on the cells and an adequate transfer of nutrients and wastes. Because the adhesive patterns and matrix formation are not currently known, initial studies determined the exact rotational forces. Cell-polymer specimens are harvested at 1, 2, 3, 6, 9, 12, 24 weeks for each polymer type and cell concentration. Specimens are collected and processed for histology, scanning and transmission electron microscopy and immunohistochemistry.

Infection of the TE Model with Influenza A Virus

If all, or at least most of the three above criteria are met, we can assess the ability of the TE bronchiole-alveolar model to support influenza A virus infection. Infectious influenza A virions are obtained by egg culture of influenza A seed stocks as previously described. Normally a single TE lung culture will yield from 8-16 individual organoids. Due to the inability to count the cells that form the individual organoids we use the weight of the TE sample to divide the culture into sham and virus exposure groups. The TE organoids (murine or human) will be exposed to the infectious virus in a plastic petri dish for 1 hour at 37° C. at MOIs from 0.5-2. After virus exposure the organoids will be gently washed with warm RPMI 1640 with 10% defined calf serum and returned to a 10 mL bioreactor chamber. Exposures (sham or virus) are also set up as duplicate or triplicate cultures and evaluations of the amount of cell/virus products are determined for each individual sample. The final measurements will be presented as averages of the duplicate or triplicate samples.

### Identifying and Characterizing the Influenza Infection of the TE Model

The presence or absence of influenza infections of the TE model will be screened using immunocytochemical labeling of viral proteins as well as immunoprecipitation followed by SDS PAGE gel electrophoresis or by western blotting. When influenza infection is detected, then the model system is evaluated for number of characteristics.

Individual influenza A products are examined and the cell types producing each product are determined by two color labeling of cell type and virus product using antibodies to each viral protein including Matrix (M1), Neuraminidase (N), Hemagglutinin (H), Nucleoprotein (NP), and the polymerase components (PB1, PA and PB2) and Non structural protein (M2). The precise cellular localization of each viral protein product are identified using monoclonal or polyclonal antibodies.

The results of the influenza infection analyses are compared to the known biology of productive and non-productive influenza A infections of lung epithelium in vivo to determine the validity of the TE models for subsequent influenza A pathogenesis studies. It is hoped that the model will be able to support the productive replication of influenza A virus infection perhaps even generating histopathologic changes in the lung tissue resembling what occurs in in vivo infection.

Individual lung epithelial protein products such as those listed in Table 2 are examined in sham exposed and virus exposed TE cultures.

### Validation of the TE Models

The TE lung and mixed TE lung/lymphatic models are designed to provide an in vitro platform for studying infection and pathogenesis of pathogens such as the influenza A virus. Validation of the TE murine model requires a comparison between (native lung) and murine in vitro TE lung, with and without the addition of the same strain immune cells. For these studies mice or TE murine lung are exposed or sham exposed to mouse adapted H1N1, PR/39. Validation of the human lung model requires a comparison of primary epithelial cell culture (Cambrex Bioscience, Rockland, Md.) type I and II pneumocytes with the TE human lung. For these studies human epithelial cell cultures or human TE lung are sham exposed or exposed to human clinical isolate H1N1 A.

### Methods Used and Protocols

### Virus Infected Animal Model

Mouse-adapted influenza A/PR8/34 (H1N1) virus are kept frozen at −70° C. Immediately before infection, aliquots are thawed and diluted to a titer of 200 LD/mL with HBSS containing BSA (17%) and gentamycin (25 p g/mL). Pathogen-free, BALBc mice (Charles River, Mass.) are infected intranasally with 50 μL of diluted virus suspension under light halothane anesthesia. Infected mice are kept at 25° C. and fed at libitum in cages throughout the infection.

### Removal of Native Lungs for Examination

The lungs (murine) are perfused with normal saline through the right ventricle to wash out blood from the lung. Human lung tissue are washed with saline and thick sections and are immediately placed in culture for human infection. Aliquots of lung tissue (murine, human) are be fixed with 4% formalin and 1% glutaraldehyde in 0.1 M cacodylate buffer (pH 7.4) and maintained at 25 cm hydrostatic pressure for 5 min to maintain inflated lungs. The lung tissue is then frozen sectioned for confocal microscopic evaluation and morphology using a cryomicrotome. Sectioned tissue are then evaluated for (1) increases in cellularity as compared to normal lung sections (2) type of cells that move to site (3) protein markers used to identify specific cell types (see Table 2). Lung sections, 0.3 cm thick transverse sections are frozen or embedded in paraffin and 4-6 μm sections are stained for hematoxylin-eosin (H&E), modified Masson's trichome for collagen, specific antibodies for cluster of differentiation evaluation (CD117, 34, 4, 8, 19, 45, 31), Pro-Surfactant C, Surfactant A, Clara Cell protein 10, Acquaporin 5 (type 1 cell evaluation).

### Characterization of Murine, Human SLPCs

For evaluation of most cell surface markers such as CD34 and CD117 aliquots of 5×10⁵ cells are incubated with anti-CD34 antibody (clone 581) conjugated directly to phycoerytherin and then with anti-CD117 (clone YB5.B8) conjugated to PerCP as described by the manufacturer (Pharmingen,). Evaluation of CC10, SPA, NEUN and β-tubulin expression is performed done after fixation of cells in 2% PAF. Aliquots of 5×10⁵ cells of freshly isolated adult lung cells acquired after centrifugal-counter current elutriation will be permeabilized for 10 minutes in 0.6% n-octyl β-D-glucopyranoside (Sigma Chemical, ST Louis Mo.). Expression of CC 10 protein or other markers of mature lung are done as previously described. Corresponding immunoglobulin (IgG)-matched isotype control antibodies or for indirect antibody staining methods staining with the secondary antibody alone are used to set baseline values for analysis markers. After fixation in 2% paraformaldehyde (PAF) cells are stored at 4° C. until fluorescent microscopy and/or flow cytometric analysis is performed.

Expected Results, Problems, Anticipated and Alternative Approaches

The models of this invention are designed to show that the virus infects both type I/II pneumocytes with viral budding and specific up regulation of SPA by Type II pneumocytes. The models of this invention are also designed to allow the comparison of possible up regulation of specific protein patterns produced by specific cell types as seen during viral infection and normal lung. The models of this invention are ideally suited for evaluating particular cellular responses such as SP-A, cathelicidin, endothelin 1, and Clara cell 10 and 16 production seen during viral infection. Thus, the models are capable to determining whether the genomic profile for the proteins shown in Table 2 are upregulated as a result of viral infection. Using Tunel analysis and activation of caspase-3, the models of this invention are capable of defining the specific role of apoptosis and the time sequence involved during the process of cell infection. By comparing both human and animal responses, the sensitivity of our model system to viral infections or other types of infections can be assessed. Our model systems are also capable to monitoring and classifying the response of individual cell types to a variety of pathogen and/or toxins.

Although the model is ideal for assessing the role of a viral infection, the models are not complete, because the models are isolated from other important contributions made by a complete system such as the role the upper respiratory tract plays in neutralizing the virus. However, the uniqueness of the model systems of this invention is that they are capable of determining and defining particular cell types or group of cell types that are important to target in a treatment. By limiting particular growth factors, we are able to create the alveolar capillary interphase, which we believe plays not only an important role in the spread of the infection, but we believe is important in defining the role capillary leaking causes in the clinical picture. Our TE organoids are capable of direct comparison to in vivo animal models through the judicious addition of factors such as VEGF to encourage the development of endothelial cells in vitro.

### Inclusion of Women and Minorities

Gender, racial, or ethnic differences do not influence inclusion of tissue samples. Samples are from healthy men and woman, at rates approximating an average of the gender and racial mix of people in South Texas. No particular subpopulation is intentionally targeted.

### Inclusion of Children

When possible, we include tissues from children, but not from fetal or stillborn children. We are interested in the isolation and propagation of adult lung progenitor cells to understand their role in tissue generation and wound healing. For this reason, research materials from human subjects greater than 1 year of age are used from discarded and properly consented autopsy procedures or surgical pathology procedures.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

1: An engineered tissue system comprising of at least one tissue selected from the group consisting of lung tissue, lymphocytes or combinations thereof and at least one mixed engineered tissue comprising at least two individual engineered tissues, where said engineered tissues are generated on a scaffold in a bio-reactor under microgravity conditions sufficient to allow 3D orientation and structural differentiation.

2: The system of claim 1, wherein the individual tissues are from a single donor.

3: The system of claim 1, wherein the engineered tissues comprise organoids and/or nodes.

4: The system of claim 1, wherein the individual tissues are selected from the group consisting of (19) Keratinizing epithelial cells such as Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, and Hair matrix cell (stem cell); (20) Wet stratified barrier epithelial cells such as, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, and Urinary epithelium cell (lining urinary bladder and urinary ducts); (21) Exocrine secretory epithelial cells such as, Salivary gland mucous cell (polysaccharide-rich secretion), Salivary gland serous cell (glycoprotein enzyme-rich secretion), Von Ebner's gland cell in tongue (washes, taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), and Clara cell of lung; (22) Hormone secreting cells such as, Anterior pituitary cells (Somatotropes, Lactotropes, Thyrotropes, Gonadotropes, Corticotropes), Intermediate pituitary cell—secreting melanocyte-stimulating hormone, Magnocellular neurosecretory cells (secreting oxytocin, secreting vasopressin), Gut and respiratory tract cells secreting serotonin (secreting endorphin, secreting somatostatin, secreting gastrin, secreting secretin, secreting cholecystokinin, secreting insulin, secreting glucagon, secreting bombesin), Thyroid gland cells (thyroid epithelial cell, parafollicular cell), Parathyroid gland cells (Parathyroid chief cell, oxyphil cell), Adrenal gland cells (chromaffin cells, secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, and Mesangial cell of kidney; (23) (Gut, Exocrine Glands and Urogenital Tract) such as intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, and Epididymal basal cell; (24) Metabolism and storage cells such as Hepatocyte (liver cell), White fat cell, Brown fat cell, Liver lipocyte; (25) Barrier function cells (Lung, Gut, Exocrine Glands and Urogenital Tract) such as Type I pneumocyte (lining air space of lung), Pancreatic duct cell (centroacinar cell). Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.); (26) Epithelial cells lining closed internal body cavities such as Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear). Cell of Claudius (lining endolymphatic space of ear), Cell of Boettcher (lining endolymphatic space of ear), Choroid plexus cell (cerebrospinal fluid secretion), Pia-arachnoid squamous cell. Pigmented ciliary epithelium cell of eye, Nonpigmented ciliary epithelium cell of eye, and Corneal endothelial cell; (27) Ciliated cells with propulsive function such as Respiratory tract ciliated cell, Oviduct ciliated cell (in female), Uterine endometrial ciliated cell (in female), Rete testis cilated cell (in male), Ductulus efferens ciliated cell (in male), and Ciliated ependymal cell of central nervous system (lining brain cavities); (28) Extracellular matrix secretion cells such as Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other nonepithelial fibroblasts, Pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, and Stellate cell of perilymphatic space of ear; (29) Contractile cells such as Red skeletal muscle cell (slow), White skeletal muscle cell (fast), Intermediate skeletal muscle cell, nuclear bag cell of Muscle spindle, nuclear chain cell of Muscle spindle, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, and Red Blood Cell; (30) Blood and immune system cells such as Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, B cells, Natural killer cell, Reticulocyte, and Stem cells and committed progenitors for the blood and immune system (various types); (31) Sensory transducer cells such as Auditory inner hair cell of organ of Corti, Auditory outer hair cell of organ of Corti, Basal cell of olfactory epithelium (stem cell for olfactory neurons), Cold-sensitive primary sensory neurons, Heat-sensitive primary sensory neurons, Merkel cell of epidermis (touch sensor), Olfactory receptor neuron, Pain-sensitive primary sensory neurons (various types), Photoreceptor rod cell of eye, Photoreceptor blue-sensitive cone cell of eye, Photoreceptor green-sensitive cone cell of eye, Photoreceptor red-sensitive cone cell of eye, Proprioceptive primary sensory neurons (various types), Touch-sensitive primary sensory neurons (various types), Type I carotid body cell (blood pH sensor), Type II carotid body cell (blood pH sensor), Type I hair cell of vestibular apparatus of ear (acceleration and gravity), Type II hair cell of vestibular apparatus of ear (acceleration and gravity), and Type I taste bud cell; (32) Autonomic neuron cells such as Cholinergic neural cell (various types), Adrenergic neural cell (various types), Peptidergic neural cell (various types); (33) Sense organ and peripheral neuron supporting cells such as Inner pillar cell of organ of Corti. Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), and Enteric glial cell; (34) Central nervous system neurons and glial cells such as Astrocyte (various types), Neuron cells (large variety of types, still poorly classified), Oligodendrocyte, and Spindle neuron; (35) Lens cells such as Anterior lens epithelial cell, and Crystallin-containing lens fiber cell; (36) Pigment cells such as Melanocyte, and Retinal pigmented epithelial cell; (37) Germ cells such as Oogonium/Oocyte, Spermatid, Spermatocyte, Spermatogonium cell (stem cell for spermatocyte), and Spermatozoon; and Nurse cells such as Ovarian follicle cell, Sertoli cell (in testis), and Thymus epithelial cell, or mixtures of combinations thereof.

5. (canceled)

6: The system of claim 1, wherein the individual engineered tissues are selected from the groups consisting of lung tissue, lymphocytes or combinations thereof.

7: A method for studying tissue responses comprising the steps of:
isolating stem cells from an animal, differentiating and growing the isolated stem cells on a scaffold in a bio-reactor under microgravity conditions sufficient to allow 3D orientation and structural differentiation to form stable, fully functional individual engineered tissues,
constructing a mixed engineered tissue from two or more of the individual engineered tissues,
exposing the mixed engineered tissue and its constituent individual engineered tissues to a pathogen, a toxin and/or an environmental stress, and
monitoring the response of each tissue to the pathogen, toxin and/or environmental stress.

8: The method of claim 7, further comprising the step of:
intermittently, periodically or continuously exposing the tissues to the pathogen, toxin and/or environmental stress and
monitoring the tissues to determine longer term tissue responses.

9: A method for studying tissue responses comprising the steps of:
isolating stem cells from an animal, differentiating and growing the isolated stem cells on a scaffold in a bio-reactor under microgravity conditions sufficient to allow 3D orientation and structural differentiation to form stable, fully functional individual engineered tissues,
constructing a mixed engineered tissue from two or more of the individual engineered tissues,
exposing the mixed engineered tissue and its constituent individual engineered tissues to a pathogen and
monitoring the response of each tissue to the pathogen.

10: The method of claim 9, further comprising the steps of:
intermittently, periodically or continuously exposing the tissues to the pathogen, and monitoring the tissues to determine disease propagation and progression.

11: A method for studying a treatment comprising the steps of:
isolating stem cells from an animal, differentiating and growing the isolated stem cells on a scaffold in a bio-reactor under microgravity conditions sufficient tot allow 3D orientation and structural differentiation to form stable, fully functional individual engineered tissues,
constructing a mixed engineered tissue from two or more of the individual engineered tissues,
exposing the mixed engineered tissue and its constituent individual engineered tissues to a pathogen, a toxin and/or an environmental stress to form infected tissues, monitoring the response of each infected tissue to the pathogen, toxin and/or environmental stress,
exposing the infected tissues to a treatment, and
monitoring the response of the tissues to the treatment.

12: The method of claim 11, further comprising the steps of:
intermittently, periodically or continuously exposing the tissues to the treatment, and
monitoring the treated tissues to determine longer term treated tissue responses.

13: A method for monitoring treatments against toxins, infections, dysfunctions or diseases, where the method includes the steps of:
inducing a toxic response, infection, dysfunction and/or a disease in a engineered tissue comprising a plurality of individual engineered tissues and one or more mixed engineered tissue including two or more individual engineered tissues, where the tissues are generated on a scaffold in a bio-reactor under microgravity conditions sufficient to allow 3D orientation and structural differentiation,
administering a treatment under treating conditions and
monitoring a response of the model to the treatment,
where the treating conditions include treatment concentration, if the treatment is a pharmaceutical, treatment intensity, if the treatment is not a pharmaceutical, exposure time, medium properties or other factors that impact pathogenicity and/or toxicity.

14: An in vitro grown, engineered lung tissue comprising engineered lung tissues derived from lung resident stem or progenitor cells, which normally function in repair and homeostasis of the lung, generated on a scaffold in a bio-reactor under microgravity conditions sufficient to allow 3D orientation and structural differentiation, where the engineered tissue is are capable of being utilized an as in vitro engineered mammalian including human lung model for examining pathogensis of pathogen.

15: An ex vivo engineering lung tissue comprising immature lung cells isolated adult human stem cells from peripheral blood and grown in a rotary cell culture system that maintains the cells in a 3D orientation, where the cells are capable of differentiation into mature, fully functional lung tissue.

16: The tissue of claim 15, wherein the tissue comprises mature, fully functional engineered lung tissue.

17: The tissue of claim 15, wherein the tissue comprises artificial lung tissue organoid.

18: An implantable ex vivo grown, mature, fully lung cells, tissues or organoids comprising engineered lung tissues derived from lung resident stem or progenitor cells, which normally function in repair and homeostasis of the lung, generated on a scaffold in a bio-reactor under microgravity conditions sufficient to allow 3D orientation and structural differentiation.

19: A cell population comprising somatic lung progenitor cells capable of support lung tissue development in both in vitro and in vivo models, where the cells can be differentiated into numerous cell types that produce Clara cell protein 10 (CC10), cytokeratin, and surfactant protein C(SP-C) prior to formation of cell/polymer constructs. The present invention also provides long-term (4-8 week) in vitro stable cultures of tissue engineered lung epithelium as human lung models for pathogenesis studies using influenza A virus and/or other pathogens and/or toxins.

20: An engineered tissue for producing anti-microbial peptides called defensins and cathelicidins, which are innate immune factors present in airway surface liquid and make up part of the lung's natural defenses comprising somatic lung progenitor cells generated on a scaffold in a bio-reactor under microgravity conditions sufficient to allow 3D orientation and structural differentiation to form several different cell types in the lung and respiratory tract including airway epithelial cells, macrophages and neutrophils which produce prostaglandins, cyclooxygenase and lipoxygenase products and de novo metabolism of arachidonic acid to both cyclooxygenase and lipoxygenase products and the production of leukotrienes is dependent on both time in culture and agonist.
